SEQUENCE LISTING (17 sequences)

SEQ ID NO: 1 - DNA, 1296 bp, Homo sapiens
atgcccaaccccaggcctggcaagccctcggccccttccttggcccttggcccatccccaggagcctcgcccagctggagggctgcacccaaagcctcagacctgctgggggcccggggcccagggggaaccttccagggccgagatcttcgaggcggggcccatgcctcctcttcttccttgaaccccatgccaccatcgcagctgcagctgcccacactgcccctagtcatggtggcaccctccggggcacggctgggccccttgccccacttacaggcactcctccaggacaggccacatttcatgcaccagctctcaacggtggatgcccacgcccggacccctgtgctgcaggtgcaccccctggagagcccagccatgatcagcctcacaccacccaccaccgccactggggtcttctccctcaaggcccggcctggcctcccacctgggatcaacgtggccagcctggaatgggtgtccagggagccggcactgctctgcaccttcccaaatcccagtgcacccaggaaggacagcaccctttcggctgtgccccagagctcctacccactgctggcaaatggtgtctgcaagtggcccggatgtgagaaggtcttcgaagagccagaggacttcctcaagcactgccaggcggaccatcttctggatgagaagggcagggcacaatgtctcctccagagagagatggtacagtctctggagcagcagctggtgctggagaaggagaagctgagtgccatgcaggcccacctggctgggaaaatggcactgaccaaggcttcatctgtggcatcatccgacaagggctcctgctgcatcgtagctgctggcagccaaggccctgtcgtcccagcctggtctggcccccgggaggcccctgacagcctgtttgctgtccggaggcacctgtggggtagccatggaaacagcacattcccagagttcctccacaacatggactacttcaagttccacaacatgcgaccccctttcacctacgccacgctcatccgctgggccatcctggaggctccagagaagcagcggacactcaatgagatctaccactggttcacacgcatgtttgccttcttcagaaaccatcctgccacctggaagaacgccatccgccacaacctgagtctgcacaagtgctttgtgcgggtggagagcgagaagggggctgtgtggaccgtggatgagctggagttccgcaagaaacggagccagaggcccagcaggtgttccaaccctacacctggcccctga

SEQ ID NO: 2 - protein, 431 aa, Homo sapiens
MPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPSRCSNPTPGP

SEQ ID NO: 3 - DNA, 232 bp, Homo sapiens
cctgcccttggacaaggacccgatgcccaaccccaggcctggcaagccctcggccccttccttggcccttggcccatccccaggagcctcgcccagctggagggctgcacccaaagcctcagacctgctgggggcccggggcccagggggaaccttccagggccgagatcttcgaggcggggcccatgcctcctcttcttccttgaaccccatgccaccatcgcagctgcag

SEQ ID NO: 4 - DNA, 1290 bp, Mus musculus
atgcccaaccctaggccagccaagcctatggctccttccttggcccttggcccatccccaggagtcttgccaagctggaagactgcacccaagggctcagaacttctagggaccaggggctctgggggacccttccaaggtcgggacctgcgaagtggggcccacacctcttcttccttgaaccccctgccaccatcccagctgcagctgcctacagtgcccctagtcatggtggcaccgtctggggcccgactaggtccctcaccccacctacaggcccttctccaggacagaccacacttcatgcatcagctctccactgtggatgcccatgcccagacccctgtgctccaagtgcgtccactggacaacccagccatgatcagcctcccaccaccttctgctgccactggggtcttctccctcaaggcccggcctggcctgccacctgggatcaatgtggccagtctggaatgggtgtccagggagccagctctactctgcaccttcccacgctcgggtacacccaggaaagacagcaaccttttggctgcaccccaaggatcctacccactgctggcaaatggagtctgcaagtggcctggttgtgagaaggtcttcgaggagccagaagagtttctcaagcactgccaagcagatcatctcctggatgagaaaggcaaggcccagtgcctcctccagagagaagtggtgcagtctctggagcagcagctggagctggaaaaggagaagctgggagctatgcaggcccacctggctgggaagatggcgctggccaaggctccatctgtggcctcaatggacaagagctcttgctgcatcgtagccaccagtactcagggcagtgtgctcccggcctggtctgctcctcgggaggctccagacggcggcctgtttgcagtgcggaggcacctctggggaagccatggcaatagttccttcccagagttcttccacaacatggactacttcaagtaccacaatatgcgaccccctttcacctatgccacccttatccgatgggccatcctggaagccccggagaggcagaggacactcaatgaaatctaccattggtttactcgcatgttcgcctacttcagaaaccaccccgccacctggaagaatgccatccgccacaacctgagcctgcacaagtgctttgtgcgagtggagagcgagaagggagcagtgtggaccgtagatgaatttgagtttcgcaagaagaggagccaacgccccaacaagtgctccaatccctgcccttga

SEQ ID NO: 5 - protein, 429 aa, Mus musculus
MPNPRPAKPMAPSLALGPSPGVLPSWKTAPKGSELLGTRGSGGPFQGRDLRSGAHTSSSLNPLPPSQLQLPTVPLVMVAPSGARLGPSPHLQALLQDRPHFMHQLSTVDAHAQTPVLQVRPLDNPAMISLPPPSAATGVFSLKARPGLPPGINVASLEWVSREPALLCTFPRSGTPRKDSNLLAAPQGSYPLLANGVCKWPGCEKVFEEPEEFLKHCQADHLLDEKGKAQCLLQREVVQSLEQQLELEKEKLGAMQAHLAGKMALAKAPSVASMDKSSCCIVATSTQGSVLPAWSAPREAPDGGLFAVRRHLWGSHGNSSFPEFFHNMDYFKYHNMRPPFTYATLIRWAILEAPERQRTLNEIYHWFTRMFAYFRNHPATWKNAIRHNLSLHKCFVRVESEKGAVWTVDEFEFRKKRSQRPNKCSNPCP

SEQ ID NO: 6 - DNA, 1295 bp, Rattus Rattus
atgcccaacccaaggccagctaagcctatggctccttccttggcccctggcccgtccccaggaggcttgccaagctggaagactgcgcccaagggctcagaactcctagggaccaggggtcctgggggacccttccaaggccgggacctgcgaagtggggcccacacctcctcttcttccttgaaccccctgccaccatcacagctgcagctgcctacagtgcccctagtcatggtggcaccctctggggcccgactaggcccctcaccgcacctgcaggcacttctccaggacagaccacactttatgcatcagctctccactgtagacgcacatggccacacccctgtgctacaagtgcgcccactggacaacccagcgatgatcggcctcccaccgcctactgctgccactggagtcttctccctcaaggcccggcctggcctgccacctgggatcaatgtggccagtctggaatgggtgtccagggagccggctctactctgcaccttcccacgctcaggtacacccaggaaagacagcaaccttctggctgcaccacaaggatcctacccactgctggcaaacggagtctgcaagtggccgggttgtgagaaggccttcgaggagccgggagagtttctcaagcactgccaagcagatcacctcttggatgagaagggcaaggcccagtgcctcctccagagagaagtggtgcagtctctggagcagcagctggagctggaaaaggagaagctgggagctatgcaggcccacctggctgggaagatggcattgacaaaagctccacctgtggcctcagtggacaagagctcctgctgcctcgtagccaccagcacccagggcagcgtcctcccggcctggtctagtccccgggaagcttcagacagcttgtttgctgtgcggagacacctctggggaagccatggaaacagcacctttccagagttcttccacaacatggactacttcaagtaccataatatgcggccccctttcacctatgccaccctcatccgatgggccatcctggaagctccagagaggcagaggacactcaatgaaatctaccattggttcacacgcatgttcgcctacttcagaaaccaccccgccacctggaagaatgccatccgccacaacctgagcctgcacaagtgctttgtgcgagtggagagtgagaagggagcagtgtggaccgtagatgaatttgagtttcgcaagaagaggagccaacgccccagcaagtgctccaacccctgcccttgacctca

SEQ ID NO: 7 - protein, 429 aa, Rattus Rattus
MPNPRPAKPMAPSLAPGPSPGGLPSWKTAPKGSELLGTRGPGGPFQGRDLRSGAHTSSSSLNPLPPSQLQLPTVPLVMVAPSGARLGPSPHLQALLQDRPHFMHQLSTVDAHGHTPVLQVRPLDNPAMIGLPPPTAATGVFSLKARPGLPPGINVASLEWVSREPALLCTFPRSGTPRKDSNLLAAPQGSYPLLANGVCKWPGCEKAFEEPGEFLKHCQADHLLDEKGKAQCLLQREVVQSLEQQLELEKEKLGAMQAHLAGKMALTKAPPVASVDKSSCCLVATSTQGSVLPAWSSPREASDSLFAVRRHLWGSHGNSTFPEFFHNMDYFKYHNMRPPFTYATLIRWAILEAPERQRTLNEIYHWFTRMFAYFRNHPATWKNAIRHNLSLHKCFVRVESEKGAVWTVDEFEFRKKRSQRPSKCSNPCP

SEQ ID NO: 8 - DNA, 819 bp, Homo sapiens
atggattcatacctgctgatgtggggactgctcacgttcatcatggtgcctggctgccaggcagagctctgtgacgatgacccgccagagatcccacacgccacattcaaagccatggcctacaaggaaggaaccatgttgaactgtgaatgcaagagaggtttccgcagaataaaaagcgggtcactctatatgctctgtacaggaaactctagccactcgtcctgggacaaccaatgtcaatgcacaagctctgccactcggaacacaacgaaacaagtgacacctcaacctgaagaacagaaagaaaggaaaaccacagaaatgcaaagtccaatgcagccagtggaccaagcgagccttccaggtcactgcagggaacctccaccatgggaaaatgaagccacagagagaatttatcatttcgtggtggggcagatggtttattatcagtgcgtccagggatacagggctctacacagaggtcctgctgagagcgtctgcaaaatgacccacgggaagacaaggtggacccagccccagctcatatgcacaggtgaaatggagaccagtcagtttccaggtgaagagaagcctcaggcaagccccgaaggccgtcctgagagtgagacttcctgcctcgtcacaacaacagattttcaaatacagacagaaatggctgcaaccatggagacgtccatatttacaacagagtaccaggtagcagtggccggctgtgttttcctgctgatcagcgtcctcctcctgagtgggctcacctggcagcggagacagaggaagagtagaagaacaatctag

SEQ ID NO: 9 - protein, 272 aa, Homo sapiens
MDSYLLMWGLLTFIMVPGCQAELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 10 - DNA, 603 bp, Homo sapiens
atggattcatacctgctgatgtggggactgctcacgttcatcatggtgcctggctgccaggcagagctctgtgacgatgacccgccagagatcccacacgccacattcaaagccatggcctacaaggaaggaaccatgttgaactgtgaatgcaagagaggtttccgcagaataaaaagcgggtcactctatatgctctgtacaggaaactctagccactcgtcctgggacaaccaatgtcaatgcacaagctctgccactcggaacacaacgaaacaagtgacacctcaacctgaagaacagaaagaaaggaaaaccacagaaatgcaaagtccaatgcagccagtggaccaagcgagccttccaggtgaagagaagcctcaggcaagccccgaaggccgtcctgagagtgagacttcctgcctcgtcacaacaacagattttcaaatacagacagaaatggctgcaaccatggagacgtccatatttacaacagagtaccaggtagcagtggccggctgtgttttcctgctgatcagcgtcctcctcctgagtgggctcacctggcagcggagacagaggaagagtagaagaacaatctag

SEQ ID NO: 11 - protein, 200 aa, Homo sapiens
MDSYLLMWGLLTFIMVPGCQAELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGEEKPQASPEGRPESETSCLVTTTDFQIQTEMAATMETSIFTTEYQVAVAGCVFLLISVLLLSGLTWQRRQRKSRRTI

SEQ ID NO: 12 - protein, 176 aa, Homo sapiens
MDSYLLMWGLLTFIMVPGCQAELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPDFQIQTEMAATMETSIFTTEYQVAVAGCVFLLISVLLLSGLTWQRRQRKSRRTI

SEQ ID NO: 13 - DNA, 3218 bp, Homo sapiens
agtttcctggctgaacacgccagcccaatacttaaagagagcaactcctgactccgatagagactggatggacccacaagggtgacagcccaggcggaccgatcttcccatcccacatcctccggcgcgatgccaaaaagaggctgacggcaactgggccttctgcagagaaagacctccgcttcactgccccggctggtcccaagggtcaggaagatggattcatacctgctgatgtggggactgctcacgttcatcatggtgcctggctgccaggcagagctctgtgacgatgacccgccagagatcccacacgccacattcaaagccatggcctacaaggaaggaaccatgttgaactgtgaatgcaagagaggtttccgcagaataaaaagcgggtcactctatatgctctgtacaggaaactctagccactcgtcctgggacaaccaatgtcaatgcacaagctctgccactcggaacacaacgaaacaagtgacacctcaacctgaagaacagaaagaaaggaaaaccacagaaatgcaaagtccaatgcagccagtggaccaagcgagccttccaggtcactgcagggaacctccaccatgggaaaatgaagccacagagagaatttatcatttcgtggtggggcagatggtttattatcagtgcgtccagggatacagggctctacacagaggtcctgctgagagcgtctgcaaaatgacccacgggaagacaaggtggacccagccccagctcatatgcacaggtgaaatggagaccagtcagtttccaggtgaagagaagcctcaggcaagccccgaaggccgtcctgagagtgagacttcctgcctcgtcacaacaacagattttcaaatacagacagaaatggctgcaaccatggagacgtccatatttacaacagagtaccaggtagcagtggccggctgtgttttcctgctgatcagcgtcctcctcctgagtgggctcacctggcagcggagacagaggaagagtagaagaacaatctagaaaaccaaaagaacaagaatttcttggtaagaagccgggaacagacaacagaagtcatgaagcccaagtgaaatcaaaggtgctaaatggtcgcccaggagacatccgttgtgcttgcctgcgttttggaagctctgaagtcacatcacaggacacggggcagtggcaaccttgtctctatgccagctcagtcccatcagagagcgagcgctacccacttctaaatagcaatttcgccgttgaagaggaagggcaaaaccactagaactctccatcttattttcatgtatatgtgttcattaaagcatgaatggtatggaactctctccaccctatatgtagtataaagaaaagtaggtttacattcatctcattccaacttcccagttcaggagtcccaaggaaagccccagcactaacgtaaatacacaacacacacactctaccctatacaactggacattgtctgcgtggttcctttctcagccgcttctgactgctgattctcccgttcacgttgcctaataaacatccttcaagaactctgggctgctacccagaaatcattttacccttggctcaatcctctaagctaacccccttctactgagccttcagtcttgaatttctaaaaaacagaggccatggcagaataatctttgggtaacttcaaaacggggcagccaaacccatgaggcaatgtcaggaacagaaggatgaatgaggtcccaggcagagaatcatacttagcaaagttttacctgtgcgttactaattggcctctttaagagttagtttctttgggattgctatgaatgataccctgaatttggcctgcactaatttgatgtttacaggtggacacacaaggtgcaaatcaatgcgtacgtttcctgagaagtgtctaaaaacaccaaaaagggatccgtacattcaatgtttatgcaaggaaggaaagaaagaaggaagtgaagagggagaagggatggaggtcacactggtagaacgtaaccacggaaaagagcgcatcaggcctggcacggtggctcaggcctataaccccagctccctaggagaccaaggcgggagcatctcttgaggccaggagtttgagaccagcctgggcagcatagcaagacacatccctacaaaaaattagaaattggctggatgtggtggcatacgcctgtagtcctagccactcaggaggctgaggcaggaggattgcttgagcccaggagttcgaggctgcagtcagtcatgatggcaccactgcactccagcctgggcaacagagcaagatcctgtctttaaggaaaaaaagacaagatgagcataccagcagtccttgaacattatcaaaaagttcagcatattagaatcaccgggaggccttgttaaaagagttcgctgggcccatcttcagagtctctgagttgttggtctggaatagagccaaatgttttgtgtgtctaacaattcccaggtgctgttgctgctgctactattccaggaacacactttgagaaccattgtgttattgctctgcacgcccacccactctcaactcccacgaaaaaaatcaacttccagagctaagatttcggtggaagtcctggttccatatctggtgcaagatctcccctcacgaatcagttgagtcaacattctagctcaacaacatcacacgattaacattaacgaaaattattcatttgggaaactatcagccagttttcacttctgaaggggcaggagagtgttatgagaaatcacggcagttttcagcagggtccagattcagattaaataactattttctgtcatttctgtgaccaaccacatacaaacagactcatctgtgcactctccccctcccccttcaggtatatgttttctgagtaaagttgaaaagaatctcagaccagaaaatatagatatatatttaaatcttacttgagtagaactgattacgacttttgggtgttgaggggtctataagatcaaaacttttccatgataatactaagatgttatcgaccatttatctgtccttctctcaaaagtgtatggtggaattttccagaagctatgtgatacgtgatgatgtcatcactctgctgttaacatataataaatttattgctattgtttataaaagaataaatgatattttttaaaaa

SEQ ID NO: 14 - protein, 446 aa, Artificial
  Basiliximab Heavy Chain
QLQQSGTVLARPGASVKMSCKASGYSFTRYWMHWIKQRPGQGLEWIGAIYPGNSDTSYNQKFEGKAKLTAVTSASTAYMELSSLTHEDSAVYYCSRDYGYYFDFWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 210 aa, Artificial
  Basiliximab Light Chain
QIVSTQSPAIMSASPGEKVTMTCSASSSRSYMQWYQQKPGTSPKRWIYDTSKLASGVPARFSGSGSGTSYSLTISSMEAEDAATYYCHQRSSYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGE

SEQ ID NO: 16 - protein, 105 aa, Homo sapiens
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAY

SEQ ID NO: 17 - protein, 251 aa, Homo sapiens
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

(19) United States
(12) Patent Application Publication
(10) Pub. No.: US 2023/0056336 A1
(43) Pub. Date: Feb. 23, 2023

(54) METHODS AND COMPOSITIONS FOR TREATING DISEASE USING TARGETED FOXP3+CD4+ T CELLS AND CELLULAR SUICIDE AGENTS

(71) Applicant: Kyverna Therapeutics, Inc., Emeryville, CA (US)

(72) Inventors: Lih-Yun Hsu, Emeryville, CA (US); Faye Wu, Emeryville, CA (US); Jeffrey Greve, Emeryville, CA (US); Erin O'Brien, Emeryville, CA (US); Jeanne Flandez, Emeryville, CA (US); John Lee, Emeryville, CA (US)

(21) Appl. No.: 17/721,927

(22) Filed: Apr. 15, 2022

Related U.S. Application Data
(60) Provisional application No. 63/175,521, filed on Apr. 15, 2021.

(51) Int. Cl. C07K 14/725; C07K 14/705; A61P 37/02

(52) U.S. Cl. CPC C07K 14/7051; C07K 14/70596; A61P 37/02; C07K 2319/03; C07K 2319/02; C07K 2319/30

(57) ABSTRACT

This document relates to methods and materials for treating a mammal having an autoimmune disease. For example, materials and methods for producing a T cell comprising a FOXP3 polypeptide and a cellular suicide agent are provided herein. Methods and materials for treating a mammal having an autoimmune disease comprising administering to a mammal having an autoimmune disease an effective amount of a T cell are also provided herein.

### CROSS-REFERENCE TO RELATED APPLICATIONS

This application claims priority to U.S. Provisional Patent Application No. 63/175,521, filed on Apr. 15, 2021, the contents of which are incorporated herein by reference in its entirety.

### BACKGROUND

This application is related to the fields of immunology and cell therapy. Autoimmune diseases are common in the United States, with more than 20 million people suffering from one of 81 known autoimmune diseases. Regulatory T-cells (Tregs) are a subpopulation of T-cells that modulate the immune system and maintain tolerance to self-antigens. Tregs play a role in preventing or treating autoimmune disease (Sakaguchi et al., Int'l Immun. 21(10):1105-1111, 2009). FOXP3, a transcription factor expressed in Tregs, has been implicated in maintaining Treg immunosuppressive functions (Hort et al., Science 299:1057-1061, 2003). The immunosuppressive mechanisms utilized by Tregs include (i) modulation of antigen-presenting cells (e.g., through CTLA-4 expression by Tregs), (ii) depriving effector T-cells of IL-2 by CD25-mediated IL-2 consumption by Tregs, (iii) cytokine production (e.g., Treg production of immunomodulatory cytokines such as IL-10 and IL-35), and generation of extracellular adenosine through the action of Treg ecto-enzymes CD39 and CD73.

### SUMMARY

This document also provides methods and materials for introducing into a T cell (e.g., a CD4⁺ T cell, a CD4⁺CD45RA+ T cell, a CD4⁺CD62L⁺ T cell, or a central memory T cell) a first nucleic acid sequence encoding a FOXP3 polypeptide and a second nucleic acid encoding a cellular suicide agent. In some embodiments, the presence of a nucleic acid sequence encoding a FOXP3 polypeptide in a mammalian cell (e.g., any of the T cells described herein) elicits a Treg phenotype in the mammalian cell as compared to when the FOXP3 polypeptide is not present in the mammalian cell. In addition, this document provides methods and materials for treating a subject having an autoimmune disease, where the methods include administering to a subject a T cell of any one of claims 1-28; and, after a period of time, administering to the subject an effective amount of a control agent capable of interacting with a cellular suicide agent, wherein the interaction between the control agent and the cellular suicide agent reduces the number of administered T cells in the subject. The methods and materials provided herein can provide a way to enhance, stabilize, and/or reduce (when necessary) the immunosuppressive effects of a T cell in order to treat the autoimmune disease.

In one aspect, this disclosure features T cells including: a first nucleic acid sequence encoding a FOXP3 polypeptide; and a second nucleic acid sequence encoding a cellular suicide agent.

In some embodiments, the first nucleic acid sequence encoding a FOXP3 polypeptide includes a mutation in exon 2, wherein the FOXP3 polypeptide including a mutation in exon 2 results in increased nuclear localization of the FOXP3 polypeptide as compared to a FOXP3 polypeptide including an exon 2 that does not include a mutation. In some embodiments, the mutation includes deletion of exon 2.

In some embodiments, the cellular suicide agent is a CD25 polypeptide.

In some embodiments, the CD25 polypeptide is a mutant CD25 polypeptide. In some embodiments, the mutant CD25 polypeptide includes one or more amino acid substitutions, insertions, or deletions as compared to a wild type CD25 polypeptide.

In some embodiments, the mutant CD25 polypeptide includes one or more amino acid substitutions selected from the group consisting of: L42A, L42W, L2F, L42G, Y43R, L2Y, L42Y, L2I, T47D, S41N, L42V, T47S, S41Q, S41E, S41D, L42F, T47N, and L42I. In some embodiments, the mutant CD25 polypeptide includes a L42A amino acid substitution. In some embodiments, the mutant CD25 polypeptide includes a L42W amino acid substitution. In some embodiments, the mutant CD25 polypeptide includes a L2I amino acid substitution. In some embodiments, the mutant CD25 polypeptide includes a Y43R amino acid substitution. In some embodiments, the mutant CD25 polypeptide includes a L2F amino acid substitution. In some embodiments, the mutant CD25 polypeptide includes a L42G amino acid substitution. In some embodiments, the mutant CD25 polypeptide includes a L2Y amino acid substitution. In some embodiments, the mutant CD25 polypeptide includes a L42Y amino acid substitution.

In some embodiments, the mutant CD25 polypeptide has increased affinity for basiliximab as compared to a wild type CD25 polypeptide.

In some embodiments, the first nucleic acid sequence is operably linked to a promoter and the second nucleic acid sequence is operably linked to a promoter.

In some embodiments, the T cell further includes a third nucleic acid sequence encoding a receptor polypeptide. In some embodiments, the third nucleic acid sequence is operably linked to a promoter.

In some embodiments, the receptor polypeptide is a chemokine receptor polypeptide. In some embodiments, the chemokine receptor polypeptide is CCR6, CCR9, or GRP15.

In some embodiments, the T cell further includes a nucleic acid sequence encoding a binding agent. In some embodiments, the nucleic acid sequence encoding the binding agent is operably linked to a promoter.

In some embodiments, the binding agent includes an antigen-binding domain selected from the group consisting of: a Fab, a F(ab′)2 fragment, a scFV, a scAb, a dAb, a single domain heavy chain antibody, and a single domain light chain antibody.

In some embodiments, the binding agent is a chimeric antigen receptor, wherein the chimeric antigen receptor includes an extracellular domain, a transmembrane domain, and an intracellular domain, wherein the extracellular domain includes an antigen-binding domain capable of binding to an antigen on an autoimmune cell, and wherein the intracellular domain includes a cytoplasmic signaling domain and one or more co-stimulatory domains.

In some embodiments, the antigen-binding domain is a scFv that is capable of binding to the antigen on the autoimmune cell.

In some embodiments, the cytoplasmic signaling domain is a CD3 zeta domain.

In some embodiments, the co-stimulatory domain includes at least one of a CD48 domain, a 4-1BB domain, a ICOS domain, an OX40 domain, and a CD27 domain.

In another aspect, this disclosure features a composition including any of the T cells described herein.

In another aspect, this disclosure features methods of producing a T-cell population expressing an exogenous FOXP3 polypeptide and a cellular suicide agent, the method including culturing any of the T cells described herein in growth media under conditions sufficient to expand the population of T-cells.

In another aspect, this disclosure features populations of T-cells prepared by any of the methods described herein.

In another aspect, this disclosure features compositions including any of the population of T-cells described herein.

In another aspect, this disclosure features vectors including: a first nucleic acid sequence encoding a FOXP3 polypeptide; and a second nucleic acid sequence encoding a cellular suicide agent.

In some embodiments of any of the vectors described herein, the first nucleic acid sequence encoding a FOXP3 polypeptide includes a mutation in exon 2, wherein the FOXP3 polypeptide including a mutation in exon 2 results in increased nuclear localization of the FOXP3 polypeptide as compared to a FOXP3 polypeptide including an exon 2 that does not include a mutation. In some embodiments, the mutation includes deletion of exon 2.

In some embodiments of any of the vectors described herein, the cellular suicide agent is a CD25 polypeptide.

In some embodiments of any of the vectors described herein, the CD25 polypeptide is a mutant CD25 polypeptide.

In some embodiments of any of the vectors described herein, the mutant CD25 polypeptide includes one or more amino acid substitutions, insertions, or deletions as compared to a wild type CD25 polypeptide.

In some embodiments of any of the vectors described herein, the mutant CD25 polypeptide includes one or more amino acid substitutions selected from the group consisting of: L42A, L42W, L2F, L42G, Y43R, L2Y, L42Y, L2I, T47D, S41N, L42V, T47S, S41Q, S41E, S41D, L42F, T47N, and L42I. In some embodiments of any of the vectors described herein, the mutant CD25 polypeptide includes a L42A amino acid substitution. In some embodiments of any of the vectors described herein, the mutant CD25 polypeptide includes a L42W amino acid substitution. In some embodiments of any of the vectors described herein, the mutant CD25 polypeptide includes a L2I amino acid substitution. In some embodiments of any of the vectors described herein, the mutant CD25 polypeptide includes a Y43R amino acid substitution. In some embodiments of any of the vectors described herein, the mutant CD25 polypeptide includes a L2F amino acid substitution. In some embodiments of any of the vectors described herein, the mutant CD25 polypeptide includes a L42G amino acid substitution. In some embodiments of any of the vectors described herein, the mutant CD25 polypeptide includes a L2Y amino acid substitution. In some embodiments of any of the vectors described herein, the mutant CD25 polypeptide includes a L42Y amino acid substitution.

In some embodiments of any of the vectors described herein, the mutant CD25 polypeptide has increased affinity for basiliximab as compared to a wild type CD25 polypeptide.

In some embodiments of any of the vectors described herein, the first nucleic acid sequence is operably linked to a promoter and the second nucleic acid sequence is operably linked to a promoter.

In some embodiments of any of the vectors described herein, the T-cell further includes a third nucleic acid sequence encoding a receptor polypeptide. In some embodiments of any of the vectors described herein, the third nucleic acid sequence is operably linked to a promoter.

In some embodiments of any of the vectors described herein, the receptor polypeptide is a chemokine receptor polypeptide. In some embodiments of any of the vectors described herein, the chemokine receptor polypeptide is CCR6, CCR9, or GRP15.

In some embodiments of any of the vectors described herein, the vector further includes a nucleic acid sequence encoding a binding agent. In some embodiments of any of the vectors described herein, the nucleic acid sequence encoding the binding agent is operably linked to a promoter.

In some embodiments of any of the vectors described herein, the binding agent includes an antigen-binding domain is selected from the group consisting of: a Fab, a F(ab′)₂ fragment, a scFV, a scAb, a dAb, a single domain heavy chain antibody, and a single domain light chain antibody.

In some embodiments of any of the vectors described herein, the binding agent is a chimeric antigen receptor, wherein the chimeric antigen receptor includes an extracellular domain, a transmembrane domain, and an intracellular domain, wherein the extracellular domain includes an antigen-binding domain capable of binding to an antigen on an autoimmune cell, and wherein the intracellular domain includes a cytoplasmic signaling domain and one or more co-stimulatory domains.

In some embodiments of any of the vectors described herein, the antigen-binding domain is a scFv that is capable of binding to the antigen on the autoimmune cell.

In some embodiments of any of the vectors described herein, the cytoplasmic signaling domain is a CD3 zeta domain.

In some embodiments of any of the vectors described herein, the co-stimulatory domain includes at least one of a CD48 domain, a 4-1BB domain, a ICOS domain, an OX40 domain, and a CD27 domain.

In some embodiments of any of the vectors described herein, the vector is a viral vector. In some embodiments of any of the vectors described herein, the viral vector is selected from the group consisting of: a lentiviral vector, a retroviral vector, an adenoviral vector, or an adeno-associated viral (AAV) vector. In some embodiments of any of the vectors described herein, the viral vector is a lentiviral vector. In some embodiments of any of the vectors described herein, the viral vector is an AAV vector.

In another aspect, this disclosure features compositions including any of the vectors described herein.

In another aspect, this disclosure features kits including any of the compositions described herein.

In another aspect, this disclosure features methods of treating a subject, wherein the method including: administering to the subject any of the T cells described herein; and after a period of time, administering to the subject an effective amount of a control agent capable of interacting with a cellular suicide agent, wherein the interaction between the control agent and the cellular suicide agent reduces the number of administered T cells in the subject.

In some embodiments, the T cell is an autologous T cell. In some embodiments, the T cell is an allogeneic T cell. In some embodiments, the T cell is a CD4⁺ T cell or a CD4⁺/CD45RA⁺ T cell.

In some embodiments, the cellular suicide agent includes a mutant CD25 polypeptide, wherein the mutant CD25 polypeptide has increased affinity to basiliximab compared to a wild type CD25 polypeptide, and the control agent is basiliximab.

In some embodiments, basiliximab includes (i) a heavy chain including an amino acid sequence at least 80% identical to SEQ ID NO: 14, and (ii) a light chain including an amino acid sequence at least 80% identical to SEQ ID NO: 15.

In some embodiments, the subject is previously diagnosed or identified as having an autoimmune disease or disorder.

In some embodiments, the autoimmune disease or disorder is inflammatory bowel disease, lupus, rheumatoid arthritis, multiple sclerosis, insulin dependent diabetes mellitis, myasthenia gravis, Graves disease, autoimmune hemolytic anemia, autoimmune thrombocytopenia purpura, Goodpasture's syndrome, pemphigus vulgaris, acute rheumatic fever, post-streptococcal glomerulonephritis, Crohn's disease, Celiac disease, or polyarteritis nodosa.

In some embodiments, administering the T cell includes intravenous injection or intravenous infusion.

In some embodiments, the administering results in amelioration of one or more symptoms of the autoimmune disease or disorder in the subject.

In another aspect method of reducing the number of administered T cells in a subject, the method including: administering to the subject an effective amount of a control agent capable of interacting with a cellular suicide agent, wherein the interaction between the control agent and the cellular suicide agent reduces the number of administered T cells in the subject.

In some embodiments, the cellular suicide agent includes the mutant CD25 polypeptide, wherein the mutant CD25 polypeptide has increased affinity to basiliximab compared to a wild type CD25 polypeptide, and the control agent is basiliximab.

In some embodiments, basiliximab includes (i) a heavy chain including an amino acid sequence at least 80% identical to SEQ ID NO: 14, and (ii) a light chain including an amino acid sequence at least 80% identical to SEQ ID NO: 15.

In some embodiments, the mutant CD25 polypeptide includes one or more amino acid substitutions selected from the group consisting of: L42A, L42W, L2F, L42G, Y43R, L2Y, L42Y, L2I, T47D, S41N, L42V, T47S, S41Q, S41E, S41D, L42F, T47N, and L42I.

In some embodiments, the interaction between the control agent and the cellular suicide agent results in antibody-dependent cellular cytotoxicity (ADCC)-mediated killing of the administered T cells.

As used herein, “T-cell function” refers to a T cell's (e.g., any of the exemplary T cells described herein) survival, stability, and/or ability to execute its intended function. For example, a CD4⁺ T cell can have an immunosuppressive function. A CD4⁺ T cell including a nucleic acid encoding a FOXP3 polypeptide can have a FOXP3-dependent expression profile that increases the immunosuppressive function of the T cell. For example, a cell transduced with a mutated FOXP3 polypeptide as described herein can have increased expression of genes that are transcriptional targets of a FOXP3 polypeptide that can result in increased Treg cell function. In some embodiments, a T cell is considered to have Treg function if the T cell exhibits or maintains the potential to exhibit an immune suppression function (e.g., an immunosuppressive phenotype).

As used herein, the term “cell surface marker” refers to polypeptides located at or on the surface of the cell such that at least a portion of the polypeptide is located at the exterior of the cell surface. As used herein, the term “cell surface expression profile” refers to one or more polypeptides located at or on the surface of the cell such that at least a portion of the polypeptide is located at the exterior of the cell surface that indicate a cell has a particular phenotype (e.g., an immunosuppressive phenotype).

As used herein, the term “control level” refers to the level (e.g., a level in a nucleus) of a corresponding wild type polypeptide in a corresponding mammalian cell.

As used herein, the term “activation” refers to induction of a signal on an immune cell (e.g., a B cell or T cell) that to results in initiation of the immune response (e.g., T cell activation). In some cases, upon binding of an antigen to a T cell receptor (TCR) or an exogenous chimeric antigen receptor (CAR), the immune cell can undergo changes in protein expression that result in the activation of the immune response. In some cases, a TCR or CAR includes a cytoplasmic signaling sequence that can drive T cell activation. For example, upon binding of the antigen, a chimeric antigen receptor comprising an intracellular domain that includes a cytoplasmic signaling sequence (e.g., an immune-receptor tyrosine-based inhibition motifs (ITAM)) that can be phosphorylated. A phosphorylated ITAM results in the induction of a T cell activation pathway that ultimately results in a T cell immune response. Examples of ITAMs include, without limitation, CD3 gamma, CD3 delta, CD3 epsilon, TCR zeta, FcR gamma, FcR beta, CD5, CD22, CD79a, and CD66d.

As used herein, the term “stimulation” refers to stage of TCR or CAR signaling where a co-stimulatory signal can be used to achieve a robust and sustained TCR or CAR signaling response. As described herein, a co-stimulatory domain can be referred to as a signaling domain. In some cases, a signaling domain (e.g., co-stimulatory domain) can be a CD27, CD28, OX40, CD30, CD40, B7-H3, NKG2C, LIGHT, CD7, CD2, 4-1BB, or PD-1.

As used herein, the term “antibody,” “antigen-binding domain,” or “antigen-binding fragment” refers to an intact immunoglobulin or to an antigen-binding portion thereof. In some embodiments, a binding agent refers to an intact immunoglobulin or to an antigen-binding portion thereof. Antigen-binding portions may be produced by recombinant DNA techniques or by enzymatic or chemical cleavage of intact antibodies. Examples of antigen-binding portions include Fab, Fab′, F(ab′).sub.2, Fv, domain antibodies (dAbs), and complementarity determining region (CDR) fragments, single-chain antibodies (scFv), chimeric antibodies, diabodies, triabodies, tetrabodies, and polypeptides that contain at least a portion of an immunoglobulin that is sufficient to confer specific antigen-binding to the polypeptide. As used herein, the term “scFv” antibody fragments comprise the VH and VL domains of an antibody, wherein these domains are present in a single polypeptide chain. Included in the definition are single domain antibody, including camelids. In some cases, the antibody is human or humanized.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1A is a plot showing the binding affinities (K_{D}) of a mutant (L42A) CD25 polypeptide to basiliximab.

FIG. 1B is a plot showing the binding affinities (KD) of a wild type CD25 polypeptide to basiliximab.

FIG. 2A is a plot showing the binding affinities (KD) of a wild type CD25 polypeptide to human interleukin-2 (hIL2).

FIG. 2B is a plot showing the binding affinities (KD) of a mutant (L42A) CD25 polypeptide to hIL2.

### DETAILED DESCRIPTION

This document also provides methods and materials for introducing into a T cell (e.g., a CD4⁺ T cell, a CD4⁺CD45RA⁺ T cell, a CD4⁺CD62L⁺ T cell, or a central memory T cell) a first nucleic acid sequence encoding a FOXP3 polypeptide and a second nucleic acid encoding a cellular suicide agent (e.g., a mutant CD25 polypeptide). In some embodiments, the presence of a nucleic acid sequence encoding a FOXP3 polypeptide in a mammalian cell (e.g., any of the T cells described herein) elicits a Treg phenotype in the mammalian cell as compared to when the FOXP3 polypeptide is not present in the mammalian cell.

This document also provides methods and materials for treating a mammal having an autoimmune disease, where the methods include administering to the mammal an effective amount of a T cell produced using the methods described herein, and, after a period of time, administering an effective amount of a control agent that reduces the number of administered T cells within the mammal. The administration of a cellular suicide agent (e.g., a mutant CD25 polypeptide) and a control agent (i.e., that induces the cellular suicide agent) enables control over the number of administered T cells in the mammal in the event the number of administered T cells needs to be reduced. In addition, this document provides methods for reducing the number of administered T cells in a subject where the method includes administering to the subject an effective amount of a control agent capable of interacting with a cellular suicide agent, wherein the interaction between the control agent and the cellular suicide agent reduces the number of administered T cells in the subject.

Some embodiments of the methods described herein result in the reduction of the number of administered T cells by about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 98%, about 99%, or 100% of the total number of administered T cells in the subject as compared to a subject having the administered T cells but not having been administered the control agent.

### FOXP3

In some embodiments, the methods include introducing into a T-cell an effective amount of a first nucleic acid sequence encoding an exogenous FOXP3 polypeptide. In some embodiments, the presence of a first nucleic acid sequence encoding an exogenous FOXP3 polypeptide in a T-cell (e.g., any of the T-cells described herein) elicits a Treg phenotype in the T-cell as compared to when the exogenous FOXP3 polypeptide is not present or expressed in the T-cell.

As used herein, “FOXP3” refers to the FOXP3 gene that encodes a transcription factor in the Forkhead box (Fox) family of transcription factors (Sakaguchi et al., Int'l Immun. 21(10):1105-1111, 2009; Pandiyan, et al., Cytokine 76(1):13-24, 2015) or a polypeptide encoded by the FOXP3 gene or a functional fragment or variant thereof. In some embodiments, when preparing a T-cell to be used in the treatment of a mammal having an autoimmune disease or disorder by administering to the mammal the T-cell, the exogenous FOXP3 polypeptide refers to a human FOXP3 polypeptide. An example of a human FOXP3 polypeptide includes, without limitation, NCBI reference sequence: NP_054728.2, or a functional fragment thereof.

In some embodiments a first nucleic acid sequence encoding an exogenous FOXP3 (e.g., full length FOXP3) polypeptide comprises a sequence that is at least 70% (e.g., at least 75%, at least 80%, at least 85%, at least 90%, at least 95%, at least 99%, or at least 100%) identical to SEQ ID NO: 1:

ATGCCCAACCCCAGGCCTGGCAAGCCCTCGGCCCCTTCCTTGGCCCTTG
GCCCATCCCCAGGAGCCTCGCCCAGCTGGAGGGCTGCACCCAAAGCCTC
AGACCTGCTGGGGGCCCGGGGCCCAGGGGGAACCTTCCAGGGCCGAGAT
CTTCGAGGCGGGGCCCATGCCTCCTCTTCTTCCTTGAACCCCATGCCAC
CATCGCAGCTGCAGCTGCCCACACTGCCCCTAGTCATGGTGGCACCCTC
CGGGGCACGGCTGGGCCCCTTGCCCCACTTACAGGCACTCCTCCAGGAC
AGGCCACATTTCATGCACCAGCTCTCAACGGTGGATGCCCACGCCCGGA
CCCCTGTGCTGCAGGTGCACCCCCTGGAGAGCCCAGCCATGATCAGCCT
CACACCACCCACCACCGCCACTGGGGTCTTCTCCCTCAAGGCCCGGCCT
GGCCTCCCACCTGGGATCAACGTGGCCAGCCTGGAATGGGTGTCCAGGG
AGCCGGCACTGCTCTGCACCTTCCCAAATCCCAGTGCACCCAGGAAGGA
CAGCACCCTTTCGGCTGTGCCCCAGAGCTCCTACCCACTGCTGGCAAAT
GGTGTCTGCAAGTGGCCCGGATGTGAGAAGGTCTTCGAAGAGCCAGAGG
ACTTCCTCAAGCACTGCCAGGCGGACCATCTTCTGGATGAGAAGGGCAG
GGCACAATGTCTCCTCCAGAGAGAGATGGTACAGTCTCTGGAGCAGCAG
CTGGTGCTGGAGAAGGAGAAGCTGAGTGCCATGCAGGCCCACCTGGCTG
GGAAAATGGCACTGACCAAGGCTTCATCTGTGGCATCATCCGACAAGGG
CTCCTGCTGCATCGTAGCTGCTGGCAGCCAAGGCCCTGTCGTCCCAGCC
TGGTCTGGCCCCCGGGAGGCCCCTGACAGCCTGTTTGCTGTCCGGAGGC
ACCTGTGGGGTAGCCATGGAAACAGCACATTCCCAGAGTTCCTCCACAA
CATGGACTACTTCAAGTTCCACAACATGCGACCCCCTTTCACCTACGCC
ACGCTCATCCGCTGGGCCATCCTGGAGGCTCCAGAGAAGCAGCGGACAC
TCAATGAGATCTACCACTGGTTCACACGCATGTTTGCCTTCTTCAGAAA
CCATCCTGCCACCTGGAAGAACGCCATCCGCCACAACCTGAGTCTGCAC
AAGTGCTTTGTGCGGGTGGAGAGCGAGAAGGGGGCTGTGTGGACCGTGG
ATGAGCTGGAGTTCCGCAAGAAACGGAGCCAGAGGCCCAGCAGGTGTTC
CAACCCTACACCTGGCCCCTGA.

In some embodiments, a first nucleic acid sequence encoding an exogenous FOXP3 (e.g., full length FOXP3) polypeptide can comprise a codon-optimized version of SEQ ID NO: 1. For example, the codon-optimized version of the nucleic acid sequence encoding exogenous FOXP3 polypeptide can include one or more (e.g., two, three, four, five, six, seven, eight, nine, ten, eleven, twelve, thirteen, fourteen, fifteen, sixteen, seventeen, eighteen, nineteen, or twenty) nucleotide differences in the nucleic acid sequence of SEQ ID NO: 1 and still encodes for the same amino acid sequence that is encoded by the nucleic acid sequence of SEQ ID NO: 1.

In some embodiments, the amino acid sequence of an exogenous FOXP3 polypeptide is at least 80% (e.g., at least 85%, at least 90%, at least 95%, at least 98% or at least 99%) identical to SEQ ID NO: 2 (with or without the signal sequence):

MPNPRPGKPSAPSLALGPSPGASPSWRAAPKASDLLGARGPGGTFQGRD
LRGGAHASSSSLNPMPPSQLQLPTLPLVMVAPSGARLGPLPHLQALLQD
RPHFMHQLSTVDAHARTPVLQVHPLESPAMISLTPPTTATGVFSLKARP
GLPPGINVASLEWVSREPALLCTFPNPSAPRKDSTLSAVPQSSYPLLAN
GVCKWPGCEKVFEEPEDFLKHCQADHLLDEKGRAQCLLQREMVQSLEQQ
LVLEKEKLSAMQAHLAGKMALTKASSVASSDKGSCCIVAAGSQGPVVPA
WSGPREAPDSLFAVRRHLWGSHGNSTFPEFLHNMDYFKFHNMRPPFTYA
TLIRWAILEAPEKQRTLNEIYHWFTRMFAFFRNHPATWKNAIRHNLSLH
KCFVRVESEKGAVWTVDELEFRKKRSQRPSRCSNPTPGP.

In some embodiments, referring to a nucleic acid sequence encoding a FOXP3 polypeptide having a mutation in exon 2, the nucleic acid sequence corresponding to FOXP3 exon 2 is at least 80% (e.g., at least 85%, at least 90%, at least 95%, at least 99%, or 100%) identical to:

(SEQ ID NO: 3)
CCTGCCCTTGGACAAGGACCCGATGCCCAACCCCAGGCCTGGCAAGCCC
TCGGCCCCTTCCTTGGCCCTTGGCCCATCCCCAGGAGCCTCGCCCAGCT
GGAGGGCTGCACCCAAAGCCTCAGACCTGCTGGGGGCCCGGGGCCCAGG
GGGAACCTTCCAGGGCCGAGATCTTCGAGGCGGGGCCCATGCCTCCTCT
TCTTCCTTGAACCCCATGCCACCATCGCAGCTGCAG.

In some embodiments referring to a nucleic acid sequence encoding a FOXP3 polypeptide having a deleted exon 2, the nucleic acid sequence that is deleted from full length FOXP3 polypeptide (SEQ ID NO: 1) is SEQ ID NO: 3 or a fragment of SEQ ID NO: 3.

In some embodiments, when preparing a T-cell to be used in the treatment of a mammal having an autoimmune disease or disorder by administering to the mammal the T-cell, the exogenous FOXP3 polypeptide refers to a mouse FOXP3 polypeptide. An example of a mouse FOXP3 polypeptide includes, without limitation, NCBI reference sequence: NP_001186276.1 or a functional fragment thereof. In some embodiments, a first nucleic acid sequence encoding an exogenous mouse FOXP3 polypeptide comprises a sequence that is at least 70% (e.g., at least 75%, at least 80%, at least 85%, at least 90%, at least 95%, at least 99%, or at least 100%) identical to SEQ ID NO: 4:

ATGCCCAACCCTAGGCCAGCCAAGCCTATGGCTCCTTCCTTGGCCCTTG
GCCCATCCCCAGGAGTCTTGCCAAGCTGGAAGACTGCACCCAAGGGCTC
AGAACTTCTAGGGACCAGGGGCTCTGGGGGACCCTTCCAAGGTCGGGAC
CTGCGAAGTGGGGCCCACACCTCTTCTTCCTTGAACCCCCTGCCACCAT
CCCAGCTGCAGCTGCCTACAGTGCCCCTAGTCATGGTGGCACCGTCTGG
GGCCCGACTAGGTCCCTCACCCCACCTACAGGCCCTTCTCCAGGACAGA
CCACACTTCATGCATCAGCTCTCCACTGTGGATGCCCATGCCCAGACCC
CTGTGCTCCAAGTGCGTCCACTGGACAACCCAGCCATGATCAGCCTCCC
ACCACCTTCTGCTGCCACTGGGGTCTTCTCCCTCAAGGCCCGGCCTGGC
CTGCCACCTGGGATCAATGTGGCCAGTCTGGAATGGGTGTCCAGGGAGC
CAGCTCTACTCTGCACCTTCCCACGCTCGGGTACACCCAGGAAAGACAG
CAACCTTTTGGCTGCACCCCAAGGATCCTACCCACTGCTGGCAAATGGA
GTCTGCAAGTGGCCTGGTTGTGAGAAGGTCTTCGAGGAGCCAGAAGAGT
TTCTCAAGCACTGCCAAGCAGATCATCTCCTGGATGAGAAAGGCAAGGC
CCAGTGCCTCCTCCAGAGAGAAGTGGTGCAGTCTCTGGAGCAGCAGCTG
GAGCTGGAAAAGGAGAAGCTGGGAGCTATGCAGGCCCACCTGGCTGGGA
AGATGGCGCTGGCCAAGGCTCCATCTGTGGCCTCAATGGACAAGAGCTC
TTGCTGCATCGTAGCCACCAGTACTCAGGGCAGTGTGCTCCCGGCCTGG
TCTGCTCCTCGGGAGGCTCCAGACGGCGGCCTGTTTGCAGTGCGGAGGC
ACCTCTGGGGAAGCCATGGCAATAGTTCCTTCCCAGAGTTCTTCCACAA
CATGGACTACTTCAAGTACCACAATATGCGACCCCCTTTCACCTATGCC
ACCCTTATCCGATGGGCCATCCTGGAAGCCCCGGAGAGGCAGAGGACAC
TCAATGAAATCTACCATTGGTTTACTCGCATGTTCGCCTACTTCAGAAA
CCACCCCGCCACCTGGAAGAATGCCATCCGCCACAACCTGAGCCTGCAC
AAGTGCTTTGTGCGAGTGGAGAGCGAGAAGGGAGCAGTGTGGACCGTAG
ATGAATTTGAGTTTCGCAAGAAGAGGAGCCAACGCCCCAACAAGTGCTC
CAATCCCTGCCCTTGA.

In some embodiments, the amino acid sequence of an exogenous mouse FOXP3 polypeptide is at least 80% (e.g., at least 85%, at least 90%, at least 95%, at least 98% or at least 99%) identical to SEQ ID NO: 5 (with or without the signal sequence):

MPNPRPAKPMAPSLALGPSPGVLPSWKTAPKGSELLGTRGSGGPFQGRD
LRSGAHTSSSLNPLPPSQLQLPTVPLVMVAPSGARLGPSPHLQALLQDR
PHFMHQLSTVDAHAQTPVLQVRPLDNPAMISLPPPSAATGVFSLKARPG
LPPGINVASLEWVSREPALLCTFPRSGTPRKDSNLLAAPQGSYPLLANG
VCKWPGCEKVFEEPEEFLKHCQADHLLDEKGKAQCLLQREVVQSLEQQL
ELEKEKLGAMQAHLAGKMALAKAPSVASMDKSSCCIVATSTQGSVLPAW
SAPREAPDGGLFAVRRHLWGSHGNSSFPEFFHNMDYFKYHNMRPPFTYA
TLIRWAILEAPERQRTLNEIYHWFTRMFAYFRNHPATWKNAIRHNLSLH
KCFVRVESEKGAVWTVDEFEFRKKRSQRPNKCSNPCP.

In some embodiments, when preparing a T-cell to be used in the treatment of a mammal having an autoimmune disease or disorder by administering to the mammal the T-cell, the exogenous FOXP3 polypeptide refers to a rat FOXP3 polypeptide. An example of a rat FOXP3 polypeptide includes, without limitation, NCBI reference sequence: NP_001101720.1, or a functional fragment thereof. In some embodiments, a first nucleic acid sequence encoding an exogenous rat FOXP3 polypeptide comprises a sequence that is at least 70% (e.g., at least 75%, at least 80%, at least 85%, at least 90%, at least 95%, at least 99%, or at least 100%) identical to SEQ ID NO: 6:

ATGCCCAACCCAAGGCCAGCTAAGCCTATGGCTCCTTCCTTGGCCCCTG
GCCCGTCCCCAGGAGGCTTGCCAAGCTGGAAGACTGCGCCCAAGGGCTC
AGAACTCCTAGGGACCAGGGGTCCTGGGGGACCCTTCCAAGGCCGGGAC
CTGCGAAGTGGGGCCCACACCTCCTCTTCTTCCTTGAACCCCCTGCCAC
CATCACAGCTGCAGCTGCCTACAGTGCCCCTAGTCATGGTGGCACCCTC
TGGGGCCCGACTAGGCCCCTCACCGCACCTGCAGGCACTTCTCCAGGAC
AGACCACACTTTATGCATCAGCTCTCCACTGTAGACGCACATGGCCACA
CCCCTGTGCTACAAGTGCGCCCACTGGACAACCCAGCGATGATCGGCCT
CCCACCGCCTACTGCTGCCACTGGAGTCTTCTCCCTCAAGGCCCGGCCT
GGCCTGCCACCTGGGATCAATGTGGCCAGTCTGGAATGGGTGTCCAGGG
AGCCGGCTCTACTCTGCACCTTCCCACGCTCAGGTACACCCAGGAAAGA
CAGCAACCTTCTGGCTGCACCACAAGGATCCTACCCACTGCTGGCAAAC
GGAGTCTGCAAGTGGCCGGGTTGTGAGAAGGCCTTCGAGGAGCCGGGAG
AGTTTCTCAAGCACTGCCAAGCAGATCACCTCTTGGATGAGAAGGGCAA
GGCCCAGTGCCTCCTCCAGAGAGAAGTGGTGCAGTCTCTGGAGCAGCAG
CTGGAGCTGGAAAAGGAGAAGCTGGGAGCTATGCAGGCCCACCTGGCTG
GGAAGATGGCATTGACAAAAGCTCCACCTGTGGCCTCAGTGGACAAGAG
CTCCTGCTGCCTCGTAGCCACCAGCACCCAGGGCAGCGTCCTCCCGGCC
TGGTCTAGTCCCCGGGAAGCTTCAGACAGCTTGTTTGCTGTGCGGAGAC
ACCTCTGGGGAAGCCATGGAAACAGCACCTTTCCAGAGTTCTTCCACAA
CATGGACTACTTCAAGTACCATAATATGCGGCCCCCTTTCACCTATGCC
ACCCTCATCCGATGGGCCATCCTGGAAGCTCCAGAGAGGCAGAGGACAC
TCAATGAAATCTACCATTGGTTCACACGCATGTTCGCCTACTTCAGAAA
CCACCCCGCCACCTGGAAGAATGCCATCCGCCACAACCTGAGCCTGCAC
AAGTGCTTTGTGCGAGTGGAGAGTGAGAAGGGAGCAGTGTGGACCGTAG
ATGAATTTGAGTTTCGCAAGAAGAGGAGCCAACGCCCCAGCAAGTGCTC
CAACCCCTGCCCTTGACCTCA.

In some embodiments, the amino acid sequence of an exogenous rat FOXP3 polypeptide is at least 80% (e.g., at least 85%, at least 90%, at least 95%, at least 98% or at least 99%) identical to SEQ ID NO: 7 (with or without the signal sequence):

MPNPRPAKPMAPSLAPGPSPGGLPSWKTAPKGSELLGTRGPGGPFQGRD
LRSGAHTSSSSLNPLPPSQLQLPTVPLVMVAPSGARLGPSPHLQALLQD
RPHFMHQLSTVDAHGHTPVLQVRPLDNPAMIGLPPPTAATGVFSLKARP
GLPPGINVASLEWVSREPALLCTFPRSGTPRKDSNLLAAPQGSYPLLAN
GVCKWPGCEKAFEEPGEFLKHCQADHLLDEKGKAQCLLQREVVQSLEQQ
LELEKEKLGAMQAHLAGKMALTKAPPVASVDKSSCCLVATSTQGSVLPA
WSSPREASDSLFAVRRHLWGSHGNSTFPEFFHNMDYFKYHNMRPPFTYA
TLIRWAILEAPERQRTLNEIYHWFTRMFAYFRNHPATWKNAIRHNLSLH
KCFVRVESEKGAVWTVDEFEFRKKRSQRPSKCSNPCP.

As can be appreciated by those in the field, mutation of amino acid residues in FOXP3 polypeptides that are not conserved between different species is less likely to have a detrimental effect on the activity of a FOXP3 polypeptide, while mutation of amino acid residues in FOXP3 polypeptides that are conserved between different species is more likely to have a detrimental effect on the activity of a FOXP3 polypeptide.

In some embodiments, transducing a T cell with a FOXP3 polypeptide (e.g., any of the exemplary FOXP3 polypeptides described herein) can result in establishment, maintenance or enhancement of a FOXP3 polypeptide-dependent expression profile that is indicative of expression profiles seen in native Treg cells (e.g., Treg cells isolated from a healthy human). In some cases, a T cell (e.g., a CD4⁺ T cell) with a FOXP3 polypeptide-dependent expression profile can have increased immunosuppressive function. For example, a cell transduced with a mutated FOXP3 polypeptide as described herein can have increased expression of genes that are transcriptional targets of a FOXP3. Increased expression of these genes (e.g., Il-2, Ctla-4, and Tnfrsf18) can result in increased Treg cell function (e.g., inhibition of responder cell proliferation).

### Cellular Suicide Agent

Also provided herein are methods and compositions including a cellular suicide agent, where the cellular suicide agent is used to enhance, stabilize, and/or reduce (when necessary) the immunosuppressive effects of a T cell. In some embodiments, a cellular suicide agent is a CD25 polypeptide (e.g., any of the exemplary CD25 polypeptides or mutant CD25 polypeptides described herein). In some embodiments, a nucleic acid sequence encoding a cellular suicide agent (e.g., a CD25 polypeptide or mutant CD25 polypeptide) is introduced into a T cell (e.g., any of the exemplary T cells described herein). In some embodiments, a second nucleic acid sequence encoding a CD25 polypeptide is introduced into a T cell (e.g., a CD4⁺ T cell, a CD4⁺CD45RA⁺ T cell, a CD4⁺CD62L⁺ T cell, or a central memory T cell) along with a first nucleic acid encoding an exogenous FOXP3 polypeptide.

As used herein, “CD25” or “CD25 polypeptide” refers to an amino acid sequence encoded by the interleukin receptor subunit alpha (Il2ra) gene (NCBI Gene ID: 3559), or a functional fragment or variant thereof. In some embodiments, the exogenous CD25 polypeptides refers to a human CD25 polypeptide. An example of a human CD25 polypeptide includes, without limitation, NCBI reference sequences: NP_000408.1, NP_001295171.1, and NP_001295172.1, or a functional fragment or variant thereof.

In some embodiments, a nucleic acid sequence encoding an exogenous CD25 polypeptide is a sequence that is at least 70% (e.g., at least 75%, at least 80%, at least 85%, at least 90%, at least 95%, at least 99%, or at least 100%) identical to:

(SEQ ID NO: 8)
ATGGATTCATACCTGCTGATGTGGGGACTGCTCACGTTCATCATGGTGC
CTGGCTGCCAGGCAGAGCTCTGTGACGATGACCCGCCAGAGATCCCACA
CGCCACATTCAAAGCCATGGCCTACAAGGAAGGAACCATGTTGAACTGT
GAATGCAAGAGAGGTTTCCGCAGAATAAAAAGCGGGTCACTCTATATGC
TCTGTACAGGAAACTCTAGCCACTCGTCCTGGGACAACCAATGTCAATG
CACAAGCTCTGCCACTCGGAACACAACGAAACAAGTGACACCTCAACCT
GAAGAACAGAAAGAAAGGAAAACCACAGAAATGCAAAGTCCAATGCAGC
CAGTGGACCAAGCGAGCCTTCCAGGTCACTGCAGGGAACCTCCACCATG
GGAAAATGAAGCCACAGAGAGAATTTATCATTTCGTGGTGGGGCAGATG
GTTTATTATCAGTGCGTCCAGGGATACAGGGCTCTACACAGAGGTCCTG
CTGAGAGCGTCTGCAAAATGACCCACGGGAAGACAAGGTGGACCCAGCC
CCAGCTCATATGCACAGGTGAAATGGAGACCAGTCAGTTTCCAGGTGAA
GAGAAGCCTCAGGCAAGCCCCGAAGGCCGTCCTGAGAGTGAGACTTCCT
GCCTCGTCACAACAACAGATTTTCAAATACAGACAGAAATGGCTGCAAC
CATGGAGACGTCCATATTTACAACAGAGTACCAGGTAGCAGTGGCCGGC
TGTGTTTTCCTGCTGATCAGCGTCCTCCTCCTGAGTGGGCTCACCTGGC
AGCGGAGACAGAGGAAGAGTAGAAGAACAATCTAG.

In some embodiments, the CD25 polypeptide comprises a sequence that is at least 80% (e.g., at least 85%, at least 90%, at least 95%, at least 98% or at least 99%) identical to:

(SEQ ID NO: 9)
MDSYLLMWGLLTFIMVPGCQAELCDDDPPEIPHATFKAMAYKEGTMLNC
ECKRGFRRIKSGSLYMLCTGNSSHSSWDNQCQCTSSATRNTTKQVTPQP
EEQKERKTTEMQSPMQPVDQASLPGHCREPPPWENEATERIYHFVVGQM
VYYQCVQGYRALHRGPAESVCKMTHGKTRWTQPQLICTGEMETSQFPGE
EKPQASPEGRPESETSCLVTTTDFQIQTEMAATMETSIFTTEYQVAVAG
CVFLLISVLLLSGLTWQRRQRKSRRTI.

In some embodiments, the CD25 polypeptide is a mature CD25 polypeptide where the signal peptide is removed (e.g., cleaved) resulting in the mature CD25 polypeptide. In some embodiments, the CD25 polypeptide comprises a sequence that is at least 80% (e.g., at least 85%, at least 90%, at least 95%, at least 98%, or at least 99%) identical to:

(SEQ ID NO: 17)
ELCDDDPPEIPHATFKAMAYKEGTMLNCECKRGFRRIKSGSLYIVILCT
GNSSHSSWDNQCQCTSSATRNTTKQVTPQPEEQKERKTTEMQSPMQPVD
QASLPGHCREPPPWENEATERIYHFVVGQMVYYQCVQGYRALHRGPAES
VCKMTHGKTRWTQPQLICTGEMETSQFPGEEKPQASPEGRPESETSCLV
TTTDFQIQTEMAATMETSIFTTEYQVAVAGCVFLLISVLLLSGLTWQRR
QRKSRRTI

In some embodiments, a nucleic acid sequence encoding a CD25 polypeptide is a sequence that is at least 70% (e.g., at least 75%, at least 80%, at least 85%, at least 90%, at least 95%, at least 99%, or at least 100%) identical to:

(SEQ ID NO: 10)
ATGGATTCATACCTGCTGATGTGGGGACTGCTCACGTTCATCATGGTGC
CTGGCTGCCAGGCAGAGCTCTGTGACGATGACCCGCCAGAGATCCCACA
CGCCACATTCAAAGCCATGGCCTACAAGGAAGGAACCATGTTGAACTGT
GAATGCAAGAGAGGTTTCCGCAGAATAAAAAGCGGGTCACTCTATATGC
TCTGTACAGGAAACTCTAGCCACTCGTCCTGGGACAACCAATGTCAATG
CACAAGCTCTGCCACTCGGAACACAACGAAACAAGTGACACCTCAACCT
GAAGAACAGAAAGAAAGGAAAACCACAGAAATGCAAAGTCCAATGCAGC
CAGTGGACCAAGCGAGCCTTCCAGGTGAAGAGAAGCCTCAGGCAAGCCC
CGAAGGCCGTCCTGAGAGTGAGACTTCCTGCCTCGTCACAACAACAGAT
TTTCAAATACAGACAGAAATGGCTGCAACCATGGAGACGTCCATATTTA
CAACAGAGTACCAGGTAGCAGTGGCCGGCTGTGTTTTCCTGCTGATCAG
CGTCCTCCTCCTGAGTGGGCTCACCTGGCAGCGGAGACAGAGGAAGAGT
AGAAGAACAATCTAG.

In some embodiments, the CD25 polypeptide comprises a sequence that is at least 80% (e.g., at least 85%, at least 90%, at least 95%, at least 98% or at least 99%) identical to:

(SEQ ID NO: 11)
MDSYLLMWGLLTFIMVPGCQAELCDDDPPEIPHATFKAMAYKEGTMLNC
ECKRGFRRIKSGSLYMLCTGNSSHSSWDNQCQCTSSATRNTTKQVTPQP
EEQKERKTTEMQSPMQPVDQASLPGEEKPQASPEGRPESETSCLVTTTD
FQIQTEMAATMETSIFTTEYQVAVAGCVFLLISVLLLSGLTWQRRQRKS
RRTI.

In some embodiments, the CD25 polypeptide comprises a sequence that is at least 80% (e.g., at least 85%, at least 90%, at least 95%, at least 98% or at least 99%) identical to:

(SEQ ID NO: 12)
MDSYLLMWGLLTFIMVPGCQAELCDDDPPEIPHATFKAMAYKEGTMLNC
ECKRGFRRIKSGSLYMLCTGNSSHSSWDNQCQCTSSATRNTTKQVTPQP
EEQKERKTTEMQSPMQPVDQASLPDFQIQTEMAATMETSIFTTEYQVAV
AGCVFLLISVLLLSGLTWQRRQRKSRRTI.

In some embodiments, the nucleic acid sequence encoding a CD25 polypeptide is a codon-optimized version of SEQ ID NOs: 8 or 10. For example, the codon-optimized version of the nucleic acid sequence encoding a CD25 polypeptide can include one or more nucleotide differences (e.g., two, three, four, five, six, seven, eight, nine, ten, eleven, twelve, thirteen, fourteen, fifteen, sixteen, seventeen, eighteen, nineteen, or twenty) in the nucleic acid sequence of SEQ ID NOs: 8 and still encodes for the same CD25 polypeptide as SEQ ID NO: 9.

In some embodiments, a nucleic acid sequence encoding an exogenous CD25 polypeptide that is introduced into a T cell (e.g., a CD4⁺ T cell, a CD4⁺CD45RA⁺ T cell, a CD4⁺CD62L⁺ T cell, or a central memory T cell) encodes a mutant CD25 polypeptide, where the mutant CD25 polypeptide has increased affinity for basiliximab as compared to a wild type CD25 polypeptide. CD25 is the alpha-chain of the interleukin 2 (IL-2) receptor and is a marker of activated T cells. In autoimmune diseases, CD4⁺CD25⁺ Treg cells engage in active suppression of autoimmunity (See Wing et al., Clin. Immunol., 249-258 (2008)).

In some embodiments, the mutant CD25 polypeptide includes one or more amino acid substitutions, insertions, or deletions that results in increased affinity of the mutated CD25 polypeptide for basiliximab as compared to the wild type CD25 polypeptide (e.g., NCBI reference sequence: NP_000408.1/SEQ ID NO: 9). In some embodiments of any of the methods where a T cell includes a nucleic acid encoding a CD25 polypeptide (e.g., a mutant CD25 polypeptide), upon contacting the T cell with basiliximab (i.e., an antibody capable of binding to the mutant CD25 polypeptide), the T cell is targeted for removal (e.g., T cell is removed via an antibody dependent cellular cytotoxicity (ADCC)-mediated killing). Basiliximab is an anti-CD25 antibody that binds to an IL-2 binding site on CD25, thereby blocking the interaction between IL-2 and CD25 and inhibiting IL-2 mediated activation of T cells. Therefore, basiliximab can be used to block IL-2 mediated activation of T cells and thereby control the T cell-mediated immune response.

Without wishing to be bound by theory, ADCC is an immune mechanism through which Fc receptor bearing effector cells can recognize and kill antibody-coated target cells (i.e., target cells expressing antigens on their surface bound by an antibody capable of inducing ADCC) (see, e.g., Roman et al. Antibody-Dependent Cellular Cytotoxicity (ADCC), Antibody Fc, Academic Press, 2014: 1-27). Thus, in some embodiments described herein, some of the methods wherein a T cell includes a nucleic acid encoding a CD25 polypeptide (e.g., a mutant CD25 polypeptide), upon contacting the T cell with basiliximab (i.e., an antibody capable of binding to the mutant CD25 polypeptide), the T cell is targeted for removal both by blocking IL-2 mediate activation of T cells, and via antibody dependent cellular cytotoxicity (ADCC)-mediated killing.

In embodiments described herein, T-cell mediated inactivation occurs primarily due to basiliximab blocking IL-2 mediated activation of T-cells, and secondarily through T cell removal via antibody dependent cellular cytotoxicity (ADCC)-mediated killing.

In some embodiments, the mutant CD25 polypeptide can include one or more (e.g., one, two, three, four, or five) amino acid substitutions at amino acid positions 2, 41, 42, 43, and 47 of SEQ ID NO: 17. In some embodiments, the mutant CD25 polypeptide includes one or more amino acid substitutions in SEQ ID NO: 17. For example, the mutant CD25 polypeptide can include one or more amino acid substitutions (e.g., one, two, three, four, or five) in SEQ ID NO: 17 selected from the group consisting of: L42A, L42W, L2F, L42G, Y43R, L2Y, L42Y, L2I, T47D, S41N, L42V, T47S, S41Q, S41E, S41D, L42F, T47N, and L42I.

In some embodiments, the nucleic acid sequence encoding a wild type CD25 polypeptide includes a sequence that is at least 70% (e.g., at least 75%, at least 80%, at least 85%, at least 90%, at least 95%, at least 99% or 100%) identical to:

(SEQ ID NO: 13)
AGTTTCCTGGCTGAACACGCCAGCCCAATACTTAAAGAGAGCAACTCCT
GACTCCGATAGAGACTGGATGGACCCACAAGGGTGACAGCCCAGGCGGA
CCGATCTTCCCATCCCACATCCTCCGGCGCGATGCCAAAAAGAGGCTGA
CGGCAACTGGGCCTTCTGCAGAGAAAGACCTCCGCTTCACTGCCCCGGC
TGGTCCCAAGGGTCAGGAAGATGGATTCATACCTGCTGATGTGGGGACT
GCTCACGTTCATCATGGTGCCTGGCTGCCAGGCAGAGCTCTGTGACGAT
GACCCGCCAGAGATCCCACACGCCACATTCAAAGCCATGGCCTACAAGG
AAGGAACCATGTTGAACTGTGAATGCAAGAGAGGTTTCCGCAGAATAAA
AAGCGGGTCACTCTATATGCTCTGTACAGGAAACTCTAGCCACTCGTCC
TGGGACAACCAATGTCAATGCACAAGCTCTGCCACTCGGAACACAACGA
AACAAGTGACACCTCAACCTGAAGAACAGAAAGAAAGGAAAACCACAGA
AATGCAAAGTCCAATGCAGCCAGTGGACCAAGCGAGCCTTCCAGGTCAC
TGCAGGGAACCTCCACCATGGGAAAATGAAGCCACAGAGAGAATTTATC
ATTTCGTGGTGGGGCAGATGGTTTATTATCAGTGCGTCCAGGGATACAG
GGCTCTACACAGAGGTCCTGCTGAGAGCGTCTGCAAAATGACCCACGGG
AAGACAAGGTGGACCCAGCCCCAGCTCATATGCACAGGTGAAATGGAGA
CCAGTCAGTTTCCAGGTGAAGAGAAGCCTCAGGCAAGCCCCGAAGGCCG
TCCTGAGAGTGAGACTTCCTGCCTCGTCACAACAACAGATTTTCAAATA
CAGACAGAAATGGCTGCAACCATGGAGACGTCCATATTTACAACAGAGT
ACCAGGTAGCAGTGGCCGGCTGTGTTTTCCTGCTGATCAGCGTCCTCCT
CCTGAGTGGGCTCACCTGGCAGCGGAGACAGAGGAAGAGTAGAAGAACA
ATCTAGAAAACCAAAAGAACAAGAATTTCTTGGTAAGAAGCCGGGAACA
GACAACAGAAGTCATGAAGCCCAAGTGAAATCAAAGGTGCTAAATGGTC
GCCCAGGAGACATCCGTTGTGCTTGCCTGCGTTTTGGAAGCTCTGAAGT
CACATCACAGGACACGGGGCAGTGGCAACCTTGTCTCTATGCCAGCTCA
GTCCCATCAGAGAGCGAGCGCTACCCACTTCTAAATAGCAATTTCGCCG
TTGAAGAGGAAGGGCAAAACCACTAGAACTCTCCATCTTATTTTCATGT
ATATGTGTTCATTAAAGCATGAATGGTATGGAACTCTCTCCACCCTATA
TGTAGTATAAAGAAAAGTAGGTTTACATTCATCTCATTCCAACTTCCCA
GTTCAGGAGTCCCAAGGAAAGCCCCAGCACTAACGTAAATACACAACAC
ACACACTCTACCCTATACAACTGGACATTGTCTGCGTGGTTCCTTTCTC
AGCCGCTTCTGACTGCTGATTCTCCCGTTCACGTTGCCTAATAAACATC
CTTCAAGAACTCTGGGCTGCTACCCAGAAATCATTTTACCCTTGGCTCA
ATCCTCTAAGCTAACCCCCTTCTACTGAGCCTTCAGTCTTGAATTTCTA
AAAAACAGAGGCCATGGCAGAATAATCTTTGGGTAACTTCAAAACGGGG
CAGCCAAACCCATGAGGCAATGTCAGGAACAGAAGGATGAATGAGGTCC
CAGGCAGAGAATCATACTTAGCAAAGTTTTACCTGTGCGTTACTAATTG
GCCTCTTTAAGAGTTAGTTTCTTTGGGATTGCTATGAATGATACCCTGA
ATTTGGCCTGCACTAATTTGATGTTTACAGGTGGACACACAAGGTGCAA
ATCAATGCGTACGTTTCCTGAGAAGTGTCTAAAAACACCAAAAAGGGAT
CCGTACATTCAATGTTTATGCAAGGAAGGAAAGAAAGAAGGAAGTGAAG
AGGGAGAAGGGATGGAGGTCACACTGGTAGAACGTAACCACGGAAAAGA
GCGCATCAGGCCTGGCACGGTGGCTCAGGCCTATAACCCCAGCTCCCTA
GGAGACCAAGGCGGGAGCATCTCTTGAGGCCAGGAGTTTGAGACCAGCC
TGGGCAGCATAGCAAGACACATCCCTACAAAAAATTAGAAATTGGCTGG
ATGTGGTGGCATACGCCTGTAGTCCTAGCCACTCAGGAGGCTGAGGCAG
GAGGATTGCTTGAGCCCAGGAGTTCGAGGCTGCAGTCAGTCATGATGGC
ACCACTGCACTCCAGCCTGGGCAACAGAGCAAGATCCTGTCTTTAAGGA
AAAAAAGACAAGATGAGCATACCAGCAGTCCTTGAACATTATCAAAAAG
TTCAGCATATTAGAATCACCGGGAGGCCTTGTTAAAAGAGTTCGCTGGG
CCCATCTTCAGAGTCTCTGAGTTGTTGGTCTGGAATAGAGCCAAATGTT
TTGTGTGTCTAACAATTCCCAGGTGCTGTTGCTGCTGCTACTATTCCAG
GAACACACTTTGAGAACCATTGTGTTATTGCTCTGCACGCCCACCCACT
CTCAACTCCCACGAAAAAAATCAACTTCCAGAGCTAAGATTTCGGTGGA
AGTCCTGGTTCCATATCTGGTGCAAGATCTCCCCTCACGAATCAGTTGA
GTCAACATTCTAGCTCAACAACATCACACGATTAACATTAACGAAAATT
ATTCATTTGGGAAACTATCAGCCAGTTTTCACTTCTGAAGGGGCAGGAG
AGTGTTATGAGAAATCACGGCAGTTTTCAGCAGGGTCCAGATTCAGATT
AAATAACTATTTTCTGTCATTTCTGTGACCAACCACATACAAACAGACT
CATCTGTGCACTCTCCCCCTCCCCCTTCAGGTATATGTTTTCTGAGTAA
AGTTGAAAAGAATCTCAGACCAGAAAATATAGATATATATTTAAATCTT
ACTTGAGTAGAACTGATTACGACTTTTGGGTGTTGAGGGGTCTATAAGA
TCAAAACTTTTCCATGATAATACTAAGATGTTATCGACCATTTATCTGT
CCTTCTCTCAAAAGTGTATGGTGGAATTTTCCAGAAGCTATGTGATACG
TGATGATGTCATCACTCTGCTGTTAACATATAATAAATTTATTGCTATT
GTTTATAAAAGAATAAATGATATTTTTTAAAAA.

In some embodiments, a cellular suicide agent (e.g., a mutant CD25 polypeptide) may also be described or specified in terms of their binding affinity to basiliximab. In some embodiments, preferred binding affinities to basiliximab include those with a dissociation constant or K_{D} of no greater than 1 nM, 2 nM, 3 nM, 4 nM, 5 nM, 6 nM, 7 nM, 8 nM, 9 nM, 10 nM, 15 nM, 20 nM, 25 nM, 30 nM, 40 nM, 50 nM, 60 nM, 70 nM, 80 nM, 90 nM, 100 nM, 110 nM, 120 nM, 130 nM, 140 nM, 150 nM, 200 nM, 250 nM, 300 nM, 400 nM, 500 nM, 600 nM, 700 nM, 800 nM, 900 nM, 1000 nM, 1100 nM, 1200 nM, 1300 nM, 1400 nM, 1500 nM, 1600 nM, 1700 nM, 1800 nM, 1900 nM, or 2000 nM (e.g., as measured by SPR).

In some embodiments, a mutant CD25 polypeptide exhibits a binding affinity (K_{D}) to basiliximab that is between about 0.1 nM to about 10 nM (e.g., about 0.1 nM to about 9 nM, about 0.1 nM to about 8 nM, about 0.1 nM to about 7 nM, about 0.1 nM to about 6 nM, about 0.1 nM to about 5 nM, about 0.1 nM to about 4 nM, about 0.1 nM to about 3 nM, about 0.1 nM to about 2 nM, about 0.1 nM to about 1 nM, about 0.1 nM to about 0.9 nM, about 0.1 nM to about 0.8 nM, about 0.1 nM to about 0.7 nM, about 0.1 nM to about 0.6 nM, about 0.1 nM to about 0.5 nM, about 0.1 nM to about 0.4 nM, about 0.1 nM to about 0.3 nM, about 0.1 nM to about 0.2 nM, about 0.2 nM to about 10 nM, about 0.2 nM to about 9 nM, about 0.2 nM to about 8 nM, about 0.2 nM to about 7 nM, about 0.2 nM to about 6 nM, about 0.2 nM to about 5 nM, about 0.2 nM to about 4 nM, about 0.2 nM to about 3 nM, about 0.2 nM to about 2 nM, about 0.2 nM to about 1 nM, about 0.2 nM to about 0.9 nM, about 0.2 nM to about 0.8 nM, about 0.2 nM to about 0.7 nM, about 0.2 nM to about 0.6 nM, about 0.2 nM to about 0.5 nM, about 0.2 nM to about 0.4 nM, about 0.2 nM to about 0.3 nM, about 0.3 nM to about 10 nM, about 0.3 nM to about 9 nM, about 0.3 nM to about 8 nM, about 0.3 nM to about 7 nM, about 0.3 nM to about 6 nM, about 0.3 nM to about 5 nM, about 0.3 nM to about 4 nM, about 0.3 nM to about 3 nM, about 0.3 nM to about 2 nM, about 0.3 nM to about 1 nM, about 0.3 nM to about 0.9 nM, about 0.3 nM to about 0.8 nM, about 0.3 nM to about 0.7 nM, about 0.3 nM to about 0.6 nM, about 0.3 nM to about 0.5 nM, about 0.3 nM to about 0.4 nM, about 0.4 nM to about 10 nM, about 0.4 nM to about 9 nM, about 0.4 nM to about 8 nM, about 0.4 nM to about 7 nM, about 0.4 nM to about 6 nM, about 0.4 nM to about 5 nM, about 0.4 nM to about 4 nM, about 0.4 nM to about 3 nM, about 0.4 nM to about 2 nM, about 0.4 nM to about 1 nM, about 0.4 nM to about 0.9 nM, about 0.4 nM to about 0.8 nM, about 0.4 nM to about 0.7 nM, about 0.4 nM to about 0.6 nM, about 0.4 nM to about 0.5 nM, about 0.5 nM to about 10 nM, about 0.5 nM to about 9 nM, about 0.5 nM to about 8 nM, about 0.5 nM to about 7 nM, about 0.5 nM to about 6 nM, about 0.5 nM to about 5 nM, about 0.5 nM to about 4 nM, about 0.5 nM to about 3 nM, about 0.5 nM to about 2 nM, about 0.5 nM to about 1 nM, about 0.5 nM to about 0.9 nM, about 0.5 nM to about 0.8 nM, about 0.5 nM to about 0.7 nM, about 0.5 nM to about 0.6 nM, about 0.6 nM to about 10 nM, about 0.6 nM to about 9 nM, about 0.6 nM to about 8 nM, about 0.6 nM to about 7 nM, about 0.6 nM to about 6 nM, about 0.6 nM to about 5 nM, about 0.6 nM to about 4 nM, about 0.6 nM to about 3 nM, about 0.6 nM to about 2 nM, about 0.6 nM to about 1 nM, about 0.6 nM to about 0.9 nM, about 0.6 nM to about 0.8 nM, about 0.6 nM to about 0.7 nM, about 0.7 nM to about 10 nM, about 0.7 nM to about 9 nM, about 0.7 nM to about 8 nM, about 0.7 nM to about 7 nM, about 0.7 nM to about 6 nM, about 0.7 nM to about 5 nM, about 0.7 nM to about 4 nM, about 0.7 nM to about 3 nM, about 0.7 nM to about 2 nM, about 0.7 nM to about 1 nM, about 0.7 nM to about 0.9 nM, about 0.7 nM to about 0.8 nM, about 0.8 nM to about 10 nM, about 0.8 nM to about 9 nM, about 0.8 nM to about 8 nM, about 0.8 nM to about 7 nM, about 0.8 nM to about 6 nM, about 0.8 nM to about 5 nM, about 0.8 nM to about 4 nM, about 0.8 nM to about 3 nM, about 0.8 nM to about 2 nM, about 0.8 nM to about 1 nM, about 0.8 nM to about 0.9 nM, about 0.9 nM to about 10 nM, about 0.9 nM to about 9 nM, about 0.9 nM to about 8 nM, about 0.9 nM to about 7 nM, about 0.9 nM to about 6 nM, about 0.9 nM to about 5 nM, about 0.9 nM to about 4 nM, about 0.9 nM to about 3 nM, about 0.9 nM to about 2 nM, about 0.9 nM to about 1 nM, about 1 nM to about 10 nM, about 1 nM to about 9 nM, about 1 nM to about 8 nM, about 1 nM to about 7 nM, about 1 nM to about 6 nM, about 1 nM to about 5 nM, about 1 nM to about 4 nM, about 1 nM to about 3 nM, about 1 nM to about 2 nM, about 2 nM to about 10 nM, about 2 nM to about 9 nM, about 2 nM to about 8 nM, about 2 nM to about 7 nM, about 2 nM to about 6 nM, about 2 nM to about 5 nM, about 2 nM to about 4 nM, about 2 nM to about 3 nM, about 3 nM to about 10 nM, about 3 nM to about 9 nM, about 3 nM to about 8 nM, about 3 nM to about 7 nM, about 3 nM to about 6 nM, about 3 nM to about 5 nM, about 3 nM to about 4 nM, about 4 nM to about 10 nM, about 4 nM to about 9 nM, about 4 nM to about 8 nM, about 4 nM to about 7 nM, about 4 nM to about 6 nM, about 4 nM to about 5 nM, about 5 nM to about 10 nM, about 5 nM to about 9 nM, about 5 nM to about 8 nM, about 5 nM to about 7 nM, about 5 nM to about 6 nM, about 6 nM to about 10 nM, about 6 nM to about 9 nM, about 6 nM to about 8 nM, about 6 nM to about 7 nM, about 7 nM to about 10 nM, about 7 nM to about 9 nM, about 7 nM to about 8 nM, about 8 nM to about 10 nM, about 8 nM to about 9 nM, or about 9 nM to about 10 nM) (e.g., as measured by SPR).

In some embodiments, a cellular suicide agent (e.g., a mutant CD25 polypeptide) may also be described or specified in terms of their binding affinity to hIL2. In some embodiments, preferred binding affinities to hIL2 include those with a dissociation constant or K_{D} of no greater than 1 nM, 2 nM, 3 nM, 4 nM, 5 nM, 6 nM, 7 nM, 8 nM, 9 nM, 10 nM, 15 nM, 20 nM, 25 nM, 30 nM, 40 nM, 50 nM, 60 nM, 70 nM, 80 nM, 90 nM, 100 nM, 110 nM, 120 nM, 130 nM, 140 nM, 150 nM, 200 nM, 250 nM, 300 nM, 400 nM, 500 nM, 600 nM, 700 nM, 800 nM, 900 nM, 1000 nM, 1100 nM, 1200 nM, 1300 nM, 1400 nM, 1500 nM, 1600 nM, 1700 nM, 1800 nM, 1900 nM, or 2000 nM (e.g., as measured by SPR).

In some embodiments, a mutant CD25 polypeptide exhibits a binding affinity (KD) to hIL2 that is between about 0.1 nM to about 10 nM (e.g., about 0.1 nM to about 9 nM, about 0.1 nM to about 8 nM, about 0.1 nM to about 7 nM, about 0.1 nM to about 6 nM, about 0.1 nM to about 5 nM, about 0.1 nM to about 4 nM, about 0.1 nM to about 3 nM, about 0.1 nM to about 2 nM, about 0.1 nM to about 1 nM, about 0.1 nM to about 0.9 nM, about 0.1 nM to about 0.8 nM, about 0.1 nM to about 0.7 nM, about 0.1 nM to about 0.6 nM, about 0.1 nM to about 0.5 nM, about 0.1 nM to about 0.4 nM, about 0.1 nM to about 0.3 nM, about 0.1 nM to about 0.2 nM, about 0.2 nM to about 10 nM, about 0.2 nM to about 9 nM, about 0.2 nM to about 8 nM, about 0.2 nM to about 7 nM, about 0.2 nM to about 6 nM, about 0.2 nM to about 5 nM, about 0.2 nM to about 4 nM, about 0.2 nM to about 3 nM, about 0.2 nM to about 2 nM, about 0.2 nM to about 1 nM, about 0.2 nM to about 0.9 nM, about 0.2 nM to about 0.8 nM, about 0.2 nM to about 0.7 nM, about 0.2 nM to about 0.6 nM, about 0.2 nM to about 0.5 nM, about 0.2 nM to about 0.4 nM, about 0.2 nM to about 0.3 nM, about 0.3 nM to about 10 nM, about 0.3 nM to about 9 nM, about 0.3 nM to about 8 nM, about 0.3 nM to about 7 nM, about 0.3 nM to about 6 nM, about 0.3 nM to about 5 nM, about 0.3 nM to about 4 nM, about 0.3 nM to about 3 nM, about 0.3 nM to about 2 nM, about 0.3 nM to about 1 nM, about 0.3 nM to about 0.9 nM, about 0.3 nM to about 0.8 nM, about 0.3 nM to about 0.7 nM, about 0.3 nM to about 0.6 nM, about 0.3 nM to about 0.5 nM, about 0.3 nM to about 0.4 nM, about 0.4 nM to about 10 nM, about 0.4 nM to about 9 nM, about 0.4 nM to about 8 nM, about 0.4 nM to about 7 nM, about 0.4 nM to about 6 nM, about 0.4 nM to about 5 nM, about 0.4 nM to about 4 nM, about 0.4 nM to about 3 nM, about 0.4 nM to about 2 nM, about 0.4 nM to about 1 nM, about 0.4 nM to about 0.9 nM, about 0.4 nM to about 0.8 nM, about 0.4 nM to about 0.7 nM, about 0.4 nM to about 0.6 nM, about 0.4 nM to about 0.5 nM, about 0.5 nM to about 10 nM, about 0.5 nM to about 9 nM, about 0.5 nM to about 8 nM, about 0.5 nM to about 7 nM, about 0.5 nM to about 6 nM, about 0.5 nM to about 5 nM, about 0.5 nM to about 4 nM, about 0.5 nM to about 3 nM, about 0.5 nM to about 2 nM, about 0.5 nM to about 1 nM, about 0.5 nM to about 0.9 nM, about 0.5 nM to about 0.8 nM, about 0.5 nM to about 0.7 nM, about 0.5 nM to about 0.6 nM, about 0.6 nM to about 10 nM, about 0.6 nM to about 9 nM, about 0.6 nM to about 8 nM, about 0.6 nM to about 7 nM, about 0.6 nM to about 6 nM, about 0.6 nM to about 5 nM, about 0.6 nM to about 4 nM, about 0.6 nM to about 3 nM, about 0.6 nM to about 2 nM, about 0.6 nM to about 1 nM, about 0.6 nM to about 0.9 nM, about 0.6 nM to about 0.8 nM, about 0.6 nM to about 0.7 nM, about 0.7 nM to about 10 nM, about 0.7 nM to about 9 nM, about 0.7 nM to about 8 nM, about 0.7 nM to about 7 nM, about 0.7 nM to about 6 nM, about 0.7 nM to about 5 nM, about 0.7 nM to about 4 nM, about 0.7 nM to about 3 nM, about 0.7 nM to about 2 nM, about 0.7 nM to about 1 nM, about 0.7 nM to about 0.9 nM, about 0.7 nM to about 0.8 nM, about 0.8 nM to about 10 nM, about 0.8 nM to about 9 nM, about 0.8 nM to about 8 nM, about 0.8 nM to about 7 nM, about 0.8 nM to about 6 nM, about 0.8 nM to about 5 nM, about 0.8 nM to about 4 nM, about 0.8 nM to about 3 nM, about 0.8 nM to about 2 nM, about 0.8 nM to about 1 nM, about 0.8 nM to about 0.9 nM, about 0.9 nM to about 10 nM, about 0.9 nM to about 9 nM, about 0.9 nM to about 8 nM, about 0.9 nM to about 7 nM, about 0.9 nM to about 6 nM, about 0.9 nM to about 5 nM, about 0.9 nM to about 4 nM, about 0.9 nM to about 3 nM, about 0.9 nM to about 2 nM, about 0.9 nM to about 1 nM, about 1 nM to about 10 nM, about 1 nM to about 9 nM, about 1 nM to about 8 nM, about 1 nM to about 7 nM, about 1 nM to about 6 nM, about 1 nM to about 5 nM, about 1 nM to about 4 nM, about 1 nM to about 3 nM, about 1 nM to about 2 nM, about 2 nM to about 10 nM, about 2 nM to about 9 nM, about 2 nM to about 8 nM, about 2 nM to about 7 nM, about 2 nM to about 6 nM, about 2 nM to about 5 nM, about 2 nM to about 4 nM, about 2 nM to about 3 nM, about 3 nM to about 10 nM, about 3 nM to about 9 nM, about 3 nM to about 8 nM, about 3 nM to about 7 nM, about 3 nM to about 6 nM, about 3 nM to about 5 nM, about 3 nM to about 4 nM, about 4 nM to about 10 nM, about 4 nM to about 9 nM, about 4 nM to about 8 nM, about 4 nM to about 7 nM, about 4 nM to about 6 nM, about 4 nM to about 5 nM, about 5 nM to about 10 nM, about 5 nM to about 9 nM, about 5 nM to about 8 nM, about 5 nM to about 7 nM, about 5 nM to about 6 nM, about 6 nM to about 10 nM, about 6 nM to about 9 nM, about 6 nM to about 8 nM, about 6 nM to about 7 nM, about 7 nM to about 10 nM, about 7 nM to about 9 nM, about 7 nM to about 8 nM, about 8 nM to about 10 nM, about 8 nM to about 9 nM, or about 9 nM to about 10 nM) (e.g., as measured by SPR).

### Control Agent

Also provided herein are control agents, where the control agent can be used to reduce the number of administered T cells (e.g., any of the exemplary T cells described herein) in a subject. In a non-limiting example, methods provided herein include administering to the subject an effective amount of a control agent capable of interacting with a cellular suicide agent, wherein the interaction between the control agent and the cellular suicide agent reduces the number of administered T cells in the subject. In some embodiments, the interaction between the control agent and the cellular suicide agent results in antibody-dependent cellular cytotoxicity (ADCC)-mediated killing of the administered T cells.

In some embodiments, the control agent is an antigen binding fragment that is capable of binding to a CD25 polypeptide or mutant CD25 polypeptide, or functional fragment or variant thereof. In some embodiments, the antigen binding fragment is basiliximab. Basiliximab is an anti-CD25 antibody that binds to an IL-2 binding site on CD25, thereby blocking the interaction between IL-2 and CD25 and inhibiting IL-2 mediated activation of T cells (see U.S. Pat. No. 6,521,230; Simulect® (basiliximab) package insert). Basiliximab is a chimeric antibody with a human IgG1 Fc region (CH1, CH2, CH3 domains) and mouse Variable heavy (Vh) and Variable light (Vl) domains. Basiliximab has been demonstrated to inhibit T cell growth by blocking the ability of IL2 to bind to IL2RA (CD25) and activate the IL2 receptor. In some embodiments, basiliximab is used to block IL-2 mediated activation of T cells and thereby control the T cell-mediated immune response. In some embodiments, blocking IL-2 mediated activation of T cells using basiliximab results in removal of the T cell from the subject (e.g., removal through a ADCC-mediated killing).

In some embodiments, the heavy chain of Basiliximab includes a sequence that is at least 70% (e.g., at least 75%, at least 80%, at least 85%, at least 90%, at least 95%, at least 99%, or 100%) identical to:

(SEQ ID NO: 14)
QLQQSGTVLARPGASVKMSCKASGYSFTRYWMEIWIKQRPGQGLEWIGA
IYPGNSDTSYNQKFEGKAKLTAVTSASTAYMELSSLTHEDSAVYYCSRD
YGYYFDFWGQGTTLTVSSASTKGPSVFPLAPSSKSTSGGTAALGCLVKD
YFPEPVTVSWNSGALTSGVHTFPAVLQSSGLYSLSSVVTVPSSSLGTQT
YICNVNHKPSNTKVDKRVEPPKSCDKTHTCPPCPAPELLGGPSVFLFPP
KPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQP
REPQVYTLPPSRDELTKNQVSLTCLVKGFYPSDIAVEWESNGQPENNYK
TTPPVLDSDGSFFLYSKLTVDKSRWQQGNVFSCSVMHEALHNHYTQKSL
SLSPGK.

In some embodiments of any of the methods of treating of treating a mammal as described herein, the light chain of Basiliximab includes a sequence that is at least 70% (e.g., at least 75%, at least 80%, at least 85%, at least 90%, at least 95%, at least 99%, or 100%) identical to:

(SEQ ID NO: 15)
QIVSTQSPAIMSASPGEKVTMTCSASSSRSYMQWYQQKPGTSPKRWIYD
TSKLASGVPARFSGSGSGTSYSLTISSMEAEDAATYYCHQRSSYTFGGG
TKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQ
GLSSPVTKSFNRGE.

### FOXP3, Cellular Suicide Agent, and Additional Agents

This document provides methods and materials for introducing into a T cell (e.g., a CD4⁺ T cell, a CD4⁺CD45RA⁺ T cell, a CD4⁺CD62L⁺ T cell, or a central memory T cell) a first nucleic acid sequence encoding a FOXP3 polypeptide, a second nucleic acid encoding a cellular suicide agent, a nucleic acid sequence encoding a chimeric antigen receptor, and a nucleic acid sequence encoding a cytokine polypeptide, that, when present in a mammalian cell, elicits a Treg phenotype (e.g., an immunosuppressive phenotype) in the mammalian cell as compared to when the FOXP3 polypeptide is not present in the mammalian cell. In such cases, the cellular suicide agent can be used to enhance, stabilize, and/or reduce (when necessary) the immunosuppressive effects of a T cell.

This document provides methods and materials for introducing into a T cell (e.g., a CD4⁺ T cell, a CD4⁺CD45RA⁺ T cell, CD4⁺CD62L⁺ T cell, or a central memory T cell) a first nucleic acid sequence encoding a FOXP3 polypeptide (e.g., any of the exemplary FOXP3 polypeptides described herein) and a second nucleic acid encoding a cellular suicide agent (e.g., any of the cellular suicide agents described herein), and a nucleic acid sequence encoding therapeutic gene product. In such cases, the cellular suicide agent can be used to enhance, stabilize, and/or reduce (when necessary) the immunosuppressive effects of a T cell. Any appropriate therapeutic gene product that enhances the immunosuppressive effects of a T cell (e.g., a CD4⁺CD45⁺ T cell) can be used. Examples of therapeutic gene products include, without limitation, antigen or antigen-binding fragments directed to interferon alpha receptor 1 (IFNAR1), interleukin 10 (IL-10, interleukin 4 (IL-4), interleukin 13 (IL-13), interleukin 6 (IL-6), IL-6 receptor (IL-6R), and any other anti-fibrotic agent. In some embodiments, the therapeutic gene product can enhance the immunosuppressive effect of the transduced cell. For example, a therapeutic gene product can be any polypeptide or other agent that prohibits an IL-6 polypeptide from binding to an IL-6 receptor (IL-6R). In such cases, a therapeutic gene product can be an antagonist for IL-6R (e.g., an antibody or antigen-binding fragment that binds to IL-6R) and/or blocking antibody or antigen-binding fragment of IL-6 (e.g., a scFv capable of binding to IL-6). Additional examples of therapeutic gene products include, without limitation, cytokines, cytokine receptors, differentiation factors, growth factors, growth factor receptors, peptide hormones, metabolic enzymes, receptors, T cell receptors, chimeric antigen receptors (CARs), transcriptional activators, transcriptional repressors, translation activators, translational repressors, immune-receptors, apoptosis inhibitors, apoptosis inducers, immune-activators, and immune-inhibitors.

This document provides methods and materials for introducing into a T cell (e.g., a CD4⁺ T cell, a CD4⁺CD45RA⁺ T cell, CD4⁺CD62L⁺ T cell, or a central memory T cell) a first nucleic acid sequence encoding a FOXP3 polypeptide (e.g., any of the exemplary FOXP3 polypeptides described herein), a second nucleic acid encoding a cellular suicide agent (e.g., any of the cellular suicide agents described herein), and a nucleic acid sequence encoding a receptor polypeptide. In some embodiments, wherein the receptor polypeptide is a chemokine receptor polypeptide. In some embodiments, the chemokine receptor polypeptide is CCR6, CCR9 or GRP15. In some embodiments, the receptor polypeptide is a GPR15 polypeptide. In some embodiments, also provided herein are materials and methods for introducing into a T cell (e.g., a CD4⁺ T cell, a CD4⁺CD45RA⁺ T cell, CD4⁺CD62L⁺ T cell, or a central memory T cell) a first nucleic acid sequence encoding a FOXP3 polypeptide (e.g., any of the exemplary FOXP3 polypeptides described herein), a second nucleic acid encoding a cellular suicide agent (e.g., any of the cellular suicide agents described herein), a nucleic acid sequence encoding a receptor polypeptide, and a nucleic acid sequence encoding a therapeutic gene product (e.g., any of the therapeutic gene products described herein). In some embodiments, also provided herein are materials and methods for introducing into a T cell (e.g., a CD4⁺ T cell, a CD4⁺CD45RA⁺ T cell, CD4⁺CD62L⁺ T cell, or a central memory T cell) a first nucleic acid sequence encoding a FOXP3 polypeptide (e.g., any of the exemplary FOXP3 polypeptides described herein), a second nucleic acid encoding a cellular suicide agent (e.g., any of the cellular suicide agents described herein), a nucleic acid sequence encoding a receptor polypeptide, and a nucleic acid sequence encoding a binding agent (e.g., any of the exemplary binding agents described herein). In such cases, the cellular suicide agent can be used to enhance, stabilize, and/or reduce (when necessary) the immunosuppressive effects of a T cell.

This document provides methods and materials for introducing into a T cell (e.g., a CD4⁺ T cell, a CD4⁺CD45RA⁺ T cell, CD4⁺CD62L⁺ T cell, or a central memory T cell) a first nucleic acid sequence encoding a FOXP3 polypeptide (e.g., any of the exemplary FOXP3 polypeptides described herein), a second nucleic acid encoding a cellular suicide agent (e.g., any of the cellular suicide agents described herein), a nucleic acid sequence encoding a therapeutic gene product (e.g., any of the exemplary therapeutic gene products as described herein), and a nucleic acid sequence encoding a binding agent. Also provided herein are methods and materials for introducing into a T cell (e.g., a CD4⁺ T cell, a CD4⁺CD45RA⁺ T cell, CD4⁺CD62L⁺ T cell, or a central memory T cell) a first nucleic acid sequence encoding a FOXP3 polypeptide (e.g., any of the exemplary FOXP3 polypeptides described herein) and a second nucleic acid encoding a cellular suicide agent, and a nucleic acid sequence encoding a binding agent (e.g., any of the exemplary binding agents described herein). Also provided herein are methods and materials for introducing into a T cell (e.g., a CD4⁺ T cell, a CD4⁺CD45RA⁺ T cell, CD4⁺CD62L⁺ T cell, or a central memory T cell) a first nucleic acid sequence encoding a FOXP3 polypeptide (e.g., any of the exemplary FOXP3 polypeptides described herein), a second nucleic acid encoding a cellular suicide agent (e.g., any of the cellular suicide agents described herein), a nucleic acid sequence encoding a therapeutic gene product (e.g., any of the exemplary therapeutic gene products as described herein), a nucleic acid sequence encoding a binding agent (e.g., any of the exemplary binding agents described herein), and a nucleic acid sequence encoding a receptor polypeptide (e.g., any of the receptor polypeptides described herein). In such cases, the cellular suicide agent can be used to enhance, stabilize, and/or reduce (when necessary) the immunosuppressive effects of a T cell.

As used herein, “binding agent” refers to any variety of extracellular substance that binds with specificity to its cognate binding partner. In some embodiments, a cell (e.g., a CD4⁺CD45RA⁺ T cell) can be transduced with nucleic acid sequences encoding a mutated FOXP3 polypeptide as described herein, a cellular suicide agent, and a binding agent. In some embodiments, a binding agent can be any polypeptide that enhances the immunosuppressive effect of a T cell (e.g., a CD4⁺CD45RA⁺ T cell). In some embodiments, a binding agent can be a chimeric antigen receptor (CAR) as described herein where the CAR has an extracellular domain, a transmembrane domain, and an intracellular domain. In cases where the binding agent is a CAR, the extracellular domain includes a polypeptide capable of binding to a molecule found specifically on autoimmune cells or tissues. For example, the extracellular domain can include a scFV domain capable of binding to antigen on an autoimmune cell.

As used herein, the term “chimeric antigen receptor” or “CAR” refers to a chimeric antigen receptor comprising an extracellular domain, a transmembrane domain, and an intracellular domain. In some cases, the extracellular domain can comprise an antigen-binding domain as described herein. In some cases, the transmembrane domain can comprise a transmembrane domain derived from a natural polypeptide obtained from a membrane-binding or transmembrane protein. For example, a transmembrane domain can include, without limitation, a transmembrane domain from a T cell receptor alpha or beta chain, a CD3 zeta chain, a CD28 polypeptide, or a CD8 polypeptide. In some cases, the intracellular domain can comprise a cytoplasmic signaling domain as described herein. In some cases, the intracellular domain can comprise a co-stimulatory domain as described herein.

In some embodiments where the chimeric antigen receptor polypeptide includes a CD3 zeta cytoplasmic signaling domain, the CD3 zeta cytoplasmic signaling domain has an amino acid sequence that is at least 80% (e.g., at least 85%, at least 90%, at least 95%, at least 99%, or 100%) identical to NCBI Reference No: NP_932170 or a fragment thereof that has activating or stimulatory activity.

In some embodiments where the chimeric antigen receptor polypeptide includes a CD28 co-stimulatory domain, the CD28 co-stimulatory domain is at least 80% (e.g., at least 85%, 90%, 95%, 99% and 100%) identical to SEQ ID NO: 16:

IEVMYPPPYLDNEKSNGTIIHVKGKHLCPSPLFPGPSKPFWVLVVVGGV
LACYSLLVTVAFIIFWVRSKRSRLLHSDYMNMTPRRPGPTRKHYQPYAP
PRDFAAY.

In some embodiments, any of the “antigen-binding domains,” “antibodies,” “ligand binding domains,” or “binding agents” described herein can bind specifically to a target selected from the group of: CD16a, CD28, CD3 (e.g., one or more of CD3α, CD3β, CD3δ, CD3ε, and CD3γ), CD33, CD20, CD19, CD22, CD123, IL-1R, IL-1, VEGF, IL-6R, IL-4, IL-10, PDL-1, TIGIT, PD-1, TIM3, CTLA4, MICA, MICB, IL-6, IL-8, TNFα, CD26a, CD36, ULBP2, CD30, CD200, IGF-1R, MUC4AC, MUC5AC, Trop-2, CMET, EGFR, HER1, HER2, HER3, PSMA, CEA, B7H3, EPCAM, BCMA, P-cadherin, CEACAM5, a UL16-binding protein (e.g., ULBP1, ULBP2, ULBP3, ULBP4, ULBP5, and ULBP6), HLA-DR, DLL4, TYRO3, AXL, MER, CD122, CD155, PDGFDD, a ligand of TGF-β receptor II (TGF-βRII), a ligand of TGF-βRIII, a ligand of DNAM1, a ligand of NKp46, a ligand of NKp44, a ligand of NKG2D, a ligand of NK30, a ligand for a scMHCI, a ligand for a scMHCII, a ligand for a scTCR, a receptor for IL-1, a receptor for IL-2, a receptor for IL-3, a receptor for IL-7, a receptor for IL-8, a receptor for IL-10, a receptor for IL-12, a receptor for IL-15, a receptor for IL-17, a receptor for IL-18, a receptor for IL-21, a receptor for PDGF-D, a receptor for stem cell factor (SCF), a receptor for stem cell-like tyrosine kinase 3 ligand (FLT3L), a receptor for MICA, a receptor for MICB, a receptor for a ULP16-binding protein, a receptor for CD155, a receptor for CD122, and a receptor for CD28.

In some embodiments, any of the “antigen-binding domains,” “antibodies,” “ligand binding domains,” or “binding agents” further include a secretion signal peptide. For example, a nucleic acid sequence encoding a binding agent further includes a nucleic acid sequence encoding a secretion signal peptide.

### Treg and Immunosuppressive Phenotypes

This document provides methods and materials for introducing into a T cell (e.g., a CD4⁺ T cell, a CD4⁺CD45RA⁺ T cell, a CD4⁺CD62L⁺ T cell, or a central memory T cell) a first nucleic acid sequence encoding a FOXP3 polypeptide (e.g., any of the FOXP3 polypeptides described herein) and a second nucleic acid encoding a cellular suicide agent (e.g., any of the cellular suicide agents described herein), that, when present in a mammalian cell, elicits a Treg phenotype (e.g., an activated Treg phenotype) in the mammalian cell as compared to when the FOXP3 polypeptide is not present in the mammalian cell. In some embodiments, an activated Treg phenotype is induced in a T cell following introduction of a first nucleic acid sequence encoding a FOXP3 polypeptide (e.g., any of the FOXP3 polypeptides described herein) and a second nucleic acid encoding a cellular suicide agent (e.g., any of the cellular suicide agents described herein). In some embodiments, a Treg phenotype includes expression of CD4, CD25, and FOXP3. In some embodiments, a Treg phenotype includes a T cell having a cell surface expression profile of CD4⁺CD25⁺CD127^{lo/-}. Additional cell surface markers used to indicate a Treg phenotype include, without limitation, CD39 and CD73. For example, a T cell having a Treg phenotype includes a cell surface expression profile including CD4⁺CD25⁺CD127^{lo/-} CD39⁺CD73⁺. In some embodiments, a Treg phenotype includes a T cell having cell surface expression of CD4⁺CD45RA⁻. For example, a CD4⁺CD45RA⁺ T cell develops into a CD4⁺CD45RA⁻ Treg cell following transduction with a first nucleic acid sequence encoding a FOXP3 polypeptide (e.g., any of the FOXP3 polypeptides described herein) and a second nucleic acid encoding a cellular suicide agent (e.g., any of the cellular suicide agents described herein). In some embodiments, a Treg phenotype includes expression of latency-associated peptide (LAP), glycoprotein A repetitions predominant (GARP), and transforming growth factor beta 1 (TGF-β1). For example, a Treg cell can include a cell surface profile of CD4⁺CD25⁺LAP⁺, CD4⁺CD25⁺GARP⁺, or CD4⁺CD25⁺LAP⁺GARP⁺.

In some embodiments, this document provides methods and materials for introducing into a T cell (e.g., CD4⁺ T cells or CD4⁺CD45RA⁺ T cells) a first nucleic acid sequence encoding a FOXP3 polypeptide (e.g., any of the FOXP3 polypeptides described herein) and a second nucleic acid sequence encoding a cellular suicide agent (e.g., any of the cellular suicide agents described herein), that, when present in a mammalian cell, elicits a immunosuppressive phenotype in the mammalian cell as compared to when the FOXP3 polypeptide is not present in the mammalian cell. In some embodiments, a suppressive phenotype in a Treg cell is confirmed by expression of one or more of CD25, CTLA-4, and GITR. In some embodiments, an immunosuppressive phenotype in a Treg cell is confirmed by a cytokine profile. For example, an immunosuppressive phenotype is confirmed in a Treg cell by low production of IL-2, IFN-gamma, and IL-17.

In some embodiments, flow cytometry is used to assess the cell surface expression profile of a T cell transduced with a first nucleic acid sequence encoding a FOXP3 polypeptide (e.g., any of the FOXP3 polypeptides described herein) and a second nucleic acid sequence encoding a cellular suicide agent (e.g., any of the cellular suicide agents described herein). In some embodiments, intracellular staining of a FOXP3 polypeptide is detected using flow cytometry. In some embodiments, flow cytometry is used to assess expression of a FOXP3 polypeptide and a cell surface expression profile (e.g., CD4 and CD25) of a T cell (e.g., any of the exemplary T cells provided herein).

### Methods of Producing T Cells

As described herein, any appropriate method of producing a Treg cell (CD4⁺CD45RA⁻ Foxp3⁺ T cell) (e.g., T cells) comprising a FOXP3 polypeptide (e.g., any of the FOXP3 polypeptides described herein) and a cellular suicide agent can be used to generate the Treg cells as described herein. In some embodiments, a cell (e.g., a T cell) that is transduced with the nucleic acid sequences described herein is isolated from a mammal (e.g., a human) using any appropriate method (e.g., magnetic activated sorting or flow cytometry-mediated sorting). In some cases, nucleic acid sequences encoding a FOXP3 polypeptide and a cellular suicide agent can be transformed into a cell (e.g., a T cell) along with nucleic acid sequences encoding a receptor polypeptide, a therapeutic gene product and a binding agent. For example, a Treg cell can be made by transducing nucleic acid sequences encoding a FOXP3 polypeptide and a cellular suicide agent into a cell (e.g., a T cell) using a lentivirus. In another example, a Treg cell can be made by transducing nucleic acid sequences encoding a FOXP3 polypeptide, a cellular suicide agent, receptor polypeptide and a therapeutic gene product into a cell (e.g., a T cell) using a lentivirus. In yet another example, a Treg cell can be made by co-transducing nucleic acid sequences encoding a FOXP3 polypeptide, a cellular suicide agent, receptor polypeptide, a therapeutic gene product, and/or a binding agent into an immune cell (e.g., a T cell) using a lentivirus. In all cases described herein, the nucleic acid sequences are operably linked to a promoter or are operably linked to other nucleic acid sequences using a self-cleaving 2A polypeptide or IRES sequence.

Methods of introducing nucleic acids and expression vectors into a cell (e.g., an eukaryotic cell) are known in the art. Non-limiting examples of methods that can be used to introduce a nucleic acid into a cell include lipofection, transfection, electroporation, microinjection, calcium phosphate transfection, dendrimer-based transfection, cationic polymer transfection, cell squeezing, sonoporation, optical transfection, impalefection, hydrodynamic delivery, magnetofection, viral transduction (e.g., adenoviral and lentiviral transduction), and nanoparticle transfection. As used herein, “transformed” and “transduced” are used interchangeably.

In some examples, the immune cell is a T cell, and the detection of memory T cells can include, e.g., the detection of the level of expression of one or more of CD45RO, CCR7, L-selectin (CD62L), CD44, CD45RA, integrin αeβ7, CD43, CD4, CD8, CD27, CD28, IL-7Rα, CD95, IL-2Rβ, CXCR3, and LFA-1.

In some embodiments, a resting Treg cell (e.g., a CD4⁺CD45RA+Foxp3^{lo} T cell) can be converted to an activated Treg cell (CD4⁺CD45RA⁻Foxp3⁺ T cell) following transduction with a first nucleic acid sequence encoding a FOXP3 polypeptide (e.g., any of the FOXP3 polypeptides described herein) and a second nucleic acid sequence encoding a cellular suicide agent (e.g., any of the cellular suicide agents described herein). In some embodiments, a naive Treg cell (e.g., a CD4⁺CD45RA⁻Foxp3⁻ T cell) can be converted to an activated Treg cell (CD4⁺CD45RA⁻Foxp3⁺ T cell) following transduction with a first nucleic acid sequence encoding a FOXP3 polypeptide (e.g., any of the FOXP3 polypeptides described herein) and a second nucleic acid sequence encoding a cellular suicide agent (e.g., any of the cellular suicide agents described herein). In some embodiments, a non Treg cell (e.g., a CD4⁺CD45RA⁺Foxp3^{lo} T cell) can be converted to an activated Treg cell (CD4⁺CD45RA⁺Foxp3⁺ T cell) following transduction with a first nucleic acid sequence encoding a FOXP3 polypeptide (e.g., any of the FOXP3 polypeptides described herein) and a second nucleic acid sequence encoding a cellular suicide agent (e.g., any of the cellular suicide agents described herein).

### Nucleic Acids/Vectors

Also provided herein are nucleic acids sequences that encode any of the polypeptides described herein. For example, nucleic acid sequences are included that encode for a FOXP3 polypeptide, a cellular suicide agent, a receptor polypeptide, a therapeutic agent comprising a polypeptide and a binding agent comprising a polypeptide. Also provided herein are vectors that include any of the nucleic acids encoding any of the polypeptides described herein. For example, the polypeptides include, without limitation, a FOXP3 polypeptide, a cellular suicide agent, receptor polypeptide, a therapeutic agent comprising a polypeptide and a binding agent comprising a polypeptide.

Any of the vectors described herein can be an expression vector. For example, an expression vector can include a promoter sequence operably linked to the sequence encoding any of the polypeptides as described herein. Non-limiting examples of vectors include plasmids, transposons, cosmids, and viral vectors (e.g., any adenoviral vectors (e.g., pSV or pCMV vectors), adeno-associated virus (AAV) vectors, lentivirus vectors, and retroviral vectors), and any Gateway® vectors. In some cases, a vector can include sufficient cis-acting elements that supplement expression where the remaining elements needed for expression can be supplied by the host mammalian cell or in an in vitro expression system. Skilled practitioners will be capable of selecting suitable vectors and mammalian cells for making any of the T cells as described herein. Any appropriate promoter (e.g., EF1 alpha) can be operably linked to any of the nucleic acid sequences described herein. Non-limiting examples of promoters to be used in any of the vectors or constructs described herein include EF1a, SFFV, PGK, CMV, CAG, UbC, MSCV, MND, EF1a hybrid, and/or CAG hybrid. As used herein, the term “operably linked” is well known in the art and refers to genetic components that are combined such that they carry out their normal functions. For example, a gene is operably linked to a promoter when its transcription is under the control of the promoter. In another example, a nucleic acid sequence can be operably linked to other nucleic acid sequence by a self-cleaving 2A polypeptide or an internal ribosome entry site (IRES). In such cases, the self-cleaving 2A polypeptide allows the second nucleic acid to be under the control of the promoter operably linked to the first nucleic acid sequence. The nucleic acid sequences described herein can be operably linked to a promoter. In some cases, the nucleic acid sequences described herein can be operably linked to any other nucleic acid sequence described herein using a self-cleaving 2A polypeptide or IRES. In some cases, the nucleic acid sequences are all included on one vector and operably linked either to a promoter upstream of the nucleic acid sequences or operably linked to the other nucleic acid sequences through a self-cleaving 2A polypeptide or an IRES.

### Compositions

Also provided herein are compositions (e.g., pharmaceutical compositions) that include at least one of any of the polypeptides (e.g., FOXP3 polypeptides and cellular suicide agents), any of the cells, or any of the nucleic acids described herein. In some embodiments, the compositions include at least one of the any of polypeptides (e.g., FOXP3 polypeptides, cellular suicide agents, receptor polypeptides, therapeutic polypeptides, and binding agent polypeptides) described herein. In some embodiments, the composition includes a nucleic acid sequence encoding a mutant CD25 polypeptide (e.g., any of the exemplary mutant CD25 polypeptides described herein). In some embodiments, the composition includes a mutant CD25 polypeptide (e.g., any of the exemplary CD25 polypeptides described herein).

In some embodiments, the compositions include any of the cells (e.g., any of the cells described herein including any of the cells produced using any of the methods described herein). In some embodiments, the pharmaceutical compositions are formulated for different routes of administration (e.g., intravenous, subcutaneous). In some embodiments, the pharmaceutical compositions can include a pharmaceutically acceptable carrier (e.g., phosphate buffered saline).

### Kits

Also provided herein are kits that include any of T cells, vectors or polypeptides described herein, any of the compositions described herein, or any of the pharmaceutical compositions described herein. In some embodiments, the kits can include instructions for performing any of the methods described herein. In some embodiments, the kits can include at least one dose of any of the compositions (e.g., pharmaceutical compositions) described herein. In some embodiments, the kits can provide a syringe for administering any of the pharmaceutical compositions described herein.

### Cells

Also provided herein are cells (e.g., any of the exemplary cells described herein or known in the art) comprising any of the nucleic acids described herein that encode any of the polypeptides (e.g., FOXP3 polypeptides, cellular suicide agents, receptor polypeptides, therapeutic polypeptides, and binding agent polypeptides) described herein. Also provided herein are cells (e.g., any of the exemplary cells described herein or known in the art) that include any of the vectors described herein that encode any of the polypeptides (e.g., FOXP3 polypeptides, cellular suicide agents, receptor polypeptides, therapeutic polypeptides, and binding agent polypeptides) described herein.

In some embodiments, the T cell is an autologous T cell obtained from the subject (e.g., the mammal to be treated). In some embodiments, the resting Treg cell (e.g., a CD4⁺CD45RA⁺Foxp3^{lo} T cell) is an autologous resting Treg cell obtained from the subject. In some embodiments, the naive Treg cell (e.g., a CD4⁺CD45RA⁻Foxp3⁻ T cell) is an autologous naïve Treg cell obtained from the subject. In some embodiments, the non Treg cell (e.g., a CD4⁺CD45RA+Foxp3^{lo} T cell) is an autologous non Treg T cell obtained from the subject.

In some embodiments, the T cells are obtained from an allogeneic source of T cells. In some embodiments, the immune cells (e.g., NK cells) are obtained from a pluripotent stem cell via a directed differentiation protocol. In some embodiments, the immune cells are genetically genetically-engineered prior to the introduction of a first nucleic sequence and a second nucleic acid sequence.

### Methods of Treatment and Methods of Reducing the Number of Administered T Cells

Also provided herein are methods of treating a mammal (e.g., a human) having an autoimmune disease that includes (i) administering to the mammal (e.g., human) a therapeutically effective amount of a cell (e.g., a T cell) transformed with a FOXP3 polypeptide and a cellular suicide agent (e.g., any of the cellular suicide agents described herein) and (ii) after a period of time, administering a therapeutically effective amount of a control agent capable of interacting with a cellular suicide agent, wherein the interaction between the control agent and the cellular suicide agent reduces the number of administered T cells in the subject. Additional methods provided include a method of treating a mammal (e.g., a human) having an autoimmune disease that includes (i) administering to the mammal (e.g., human) a therapeutically effective amount of a cell (e.g., a T cell) transformed with a FOXP3 polypeptide, a cellular suicide agent (e.g., any of the cellular suicide agents described herein), and any combination of a therapeutic gene product polypeptide, receptor polypeptide, and a binding agent polypeptide as described herein or any of the compositions (e.g., pharmaceutical compositions) described herein and (ii) after a period of time, administering a therapeutically effective amount of a control agent capable of interacting with a cellular suicide agent, wherein the interaction between the control agent and the cellular suicide agent reduces the number of administered T cells in the subject.

As used herein, the term “control agent” refers to a molecule (e.g., a small molecule or a macromolecule (e.g., an antibody)) that induces cell death either directly or indirectly in a T cell (e.g., any of the T cells described herein).

As used herein, the term “after a period of time” can refer to 12 hours, 24 hours, 48 hours, 72 hours, 96 hours, 1 week, 2 weeks, 3 weeks, 4 weeks, 5 weeks, 6 weeks, 8 weeks, or 12 weeks, 4 months, 5 months, 6 months, 7 months, 8 months, 9 months, 10 months, 11 months, 12 months, 18 months, 2 years, 3 years, 4 years or 5 years after administration of a T cell (e.g., any of the T cells described herein).

In some embodiments of any of the methods of treating a subject described herein, the cellular suicide agent includes the mutant CD25 polypeptide, wherein the mutant CD25 polypeptide has increased affinity to basiliximab compared to a wild type CD25 polypeptide, and the control agent is basiliximab. In some embodiments, the basiliximab comprises (i) a heavy chain comprising an amino acid sequence at least 80% identical to SEQ ID NO: 14, and (ii) a light chain comprising an amino acid sequence at least 80% identical to SEQ ID NO: 15. In some embodiments, the mutant CD25 polypeptide comprises one or more amino acid substitutions in SEQ ID NO: 17. For example, the mutant CD25 polypeptide can include one or more (e.g., one, two, three, four, or five) amino acid substitutions at amino acid positions 2, 41, 42, 43, and 47 of SEQ ID NO: 17. For example, the mutant CD25 polypeptide can include one or more amino acid substitutions (e.g., one, two, three, four, or five) in SEQ ID NO: 17 selected from the group consisting of: L42A, L42W, L2F, L42G, Y43R, L2Y, L42Y, L2I, T47D, S41N, L42V, T47S, S41Q, S41E, S41D, L42F, T47N, and L42I. In some embodiments, basiliximab is administered as a bolus injection or diluted to a volume of 25 mL (10-mg vial) or 50 mL (20-mg vial) with normal saline or dextrose 5% and administered as an intravenous infusion over 20 to 30 minutes. (See Simulect® (basiliximab), Package Insert)

In some embodiments, these methods can result in a reduction in the number, severity, or frequency of one or more symptoms of the autoimmune diseases in the mammal (e.g., as compared to the number, severity, or frequency of the one or more symptoms of the autoimmune disease in the mammal prior to treatment). For example, a mammal having an autoimmune disease having been administered a T cell as described here can experience a reduction in inflammation or autoantibody production.

In some embodiments, a mammal having been administered a T cell (e.g., any of the T cells described herein), after a period of time, can be administered a therapeutically effective amount of a control agent (e.g., basiliximab) that results in the reduction in the number of administered T cells in the mammal. In some embodiments, administering, after a period of time, a therapeutically effective amount of a control agent to a mammal having been administered a T cell (e.g., any of the T cells described herein) can reduce the number of administered T cells in the mammal by at least a 5% decrease, at least a 10% decrease, at least a 15% decrease, at least a 20% decrease, at least a 25% decrease, at least a 30% decrease, at least a 35% decrease, at least a 40% decrease, at least a 45% decrease, at least a 50% decrease, at least a 55% decrease, at least a 60% decrease, at least a 65% decrease, at least a 70% decrease, at least a 75% decrease, at least a 80% decrease, at least a 85% decrease, at least a 90% decrease, at least a 95% decrease, at least a 97% decrease, at least a 98% decrease, at least a 99% decrease, (e.g., or any of the subranges of this range described herein)) from the total number of T cells administered to the mammal in the original dose.

Any appropriate method of administration can be used to administer the T cells to a mammal (e.g. a human) having an autoimmune disease. Examples of methods of administration include, without limitation, parenteral administration and intravenous injection.

Any appropriate method of administration can be used to administer the control agent to a mammal (e.g., a human) having been administered a T cell (e.g., any of the T cells described herein). Examples of methods of administration include, without limitation, parenteral administration, intravenous injection, intradermal, oral (e.g., inhalation), transdermal (e.g., topical), transmucosal, or intramuscular.

A pharmaceutical composition containing the T cells and a pharmaceutically acceptable carrier can be administered to a mammal (e.g., a human) having an autoimmune disease. For example, a pharmaceutical composition (e.g., a T cell along with a pharmaceutically acceptable carrier) to be administered to a mammal having an autoimmune disease can be formulated in an injectable form (e.g., emulsion, solution and/or suspension).

Pharmaceutically acceptable carriers, fillers, and vehicles that can be used in a pharmaceutical composition described herein can include, without limitation, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Effective dosage of T cells can vary depending on the severity of the autoimmune disease, the route of administration, the age and general health condition of the subject, excipient usage, the possibility of co-usage with other therapeutic treatments, and the judgment of the treating physician. An effective amount of a T cell can be any amount that reduces inflammation and autoantibody production within a mammal having an autoimmune disease without producing significant toxicity to the mammal. For example, an effective amount of T cells administered to a mammal having an autoimmune disease can be from about 1×10⁶ cells to about 1×10¹⁰ (e.g., from about 1×10⁶ to about 1×10⁹, from about 1×10⁶ to about 1×10⁸, from about 1×10⁶ to about 1×10⁷, from about 1×10⁷ to about 1×10¹⁰, from about 1×10⁷ to about 1×10⁹, from about 1×10⁷ to about 1×10⁸, from about 1×10⁸ to about 1×10¹⁰, from about 1×10⁸ to about 1×10⁹, or form about 1×10⁹ to about 1×10¹⁰) cells. In some cases, the T cells can be a purified population of immune cells generated as described herein. In some cases, the purity of the population of T cells can be assessed using any appropriate method, including, without limitation, flow cytometry. In some cases, the population of T cells to be administered can include a range of purities from about 70% to about 100%, from about 70% to about 90%, from about 70% to about 80%, from about 80% to about 90%, from about 90% to about 100%, from about 80% to about 100%, from about 80% to about 90%, or from about 90% to 100%. In some cases, the dosage (e.g., number of T cells to be administered) can adjusted based on the level of purity of the T cells.

Effective dosage of a control agent (e.g., Basiliximab) can vary depending on the severity of the autoimmune disease, the route of administration, the age and general health condition of the subject, excipient usage, the possibility of co-usage with other therapeutic treatments, and the judgment of the treating physician. An effective amount of a control agent (e.g., Basiliximab) can be any amount that reduces the number of targeted T cells within a mammal having been administered a T cell (e.g., any of the T cells described herein) to treat an autoimmune disease without producing significant toxicity to the mammal.

The frequency of administration of a T cell (e.g., any of the T cells described herein) can be any frequency that reduces inflammation or autoantibody production within a mammal having an autoimmune disease without producing toxicity to the mammal. In some cases, the actual frequency of administration of a T cell can vary depending on various factors including, without limitation, the effective amount, duration of treatment, use of multiple treatment agents, route of administration, and severity of the condition may require an increase or decrease in frequency of administration.

The frequency of administration of a control agent (e.g., Basiliximab) can be any frequency that reduces the number of targeted T cells within a mammal having previously been administered a T cell (e.g., any of the T cells described herein). In some cases, the actual frequency of administration of the control agent (e.g., Basiliximab) can vary depending on various factors including, without limitation, the effective amount, duration of treatment, use of multiple treatment agents, route of administration, and severity of the condition may require an increase or decrease in frequency of administration.

An effective duration for administering a composition containing a T cell can be any duration that reduces inflammation or autoantibody production within a mammal having an autoimmune disease without producing toxicity to the mammal. In some cases, the effective duration can vary from several days to several months. In general, the effective treatment duration for administering a composition containing a T cell to treat an autoimmune disease can range in duration from about one month to about five years (e.g., from about two months to about five years, from about three months to about five years, from about six months to about five years, from about eight months to about five years, from about one year to about five years, from about one month to about four years, from about one month to about three years, from about one month to about two years, from about six months to about four years, from about six months to about three years, or from about six months to about two years). In some cases, the effective treatment duration for administering a composition containing a T cell can be for the remainder of the life of the mammal.

In some cases, a course of treatment and/or the severity of one or more symptoms related to autoimmune disease can be monitored. Any appropriate method can be used to determine whether the autoimmune disease is being treated. For example, immunological techniques (e.g., ELISA) can be performed to determine if the level of autoantibodies present within a mammal being treated as described herein is reduced following the administration of the T cells. Remission and relapse of the disease can be monitored by testing for one or more markers of autoimmune disease.

Any appropriate autoimmune disease can be treated with a T cell as described herein. In some cases, an autoimmune disease caused by the accumulation of autoantibodies can be treated with a T cell as described herein. Examples of autoimmune diseases include, without limitation, inflammatory bowel disease, lupus, rheumatoid arthritis, multiple sclerosis, insulin dependent diabetes mellitis, myasthenia gravis, Grave's disease, autoimmune hemolytic anemia, autoimmune thrombocytopenia purpura, Goodpasture's syndrome, pemphigus vulgaris, acute rheumatic fever, post-streptococcal glomerulonephritis, Crohn's disease, Celiac disease, and polyarteritis nodosa.

The invention will be further described in the following examples, which do not limit the scope of the invention described in the claims.

### EXAMPLES

### Example 1. T Cell Transduced with Nucleic Acid Sequences Encoding a FOXP3 Polypeptide and a Mutant CD25 Polypeptide

A set of experiments was performed to assess the ability to co-express a cellular suicide polypeptide and a FOXP3 polypeptide in a T cell. In these experiments, CD4⁺ T cells are transduced with a lentivirus where the lentiviral vector includes a first nucleic acid sequence encoding a FOXP3 polypeptide and a second nucleic acid encoding a mutant CD25 polypeptide. The vector includes an EF1a promoter. Lentivirus is produced in HEK293 cells according to standard protocols.

CD4⁺ T cells are counted and checked for viability. Next cells are re-suspended in fresh serum free ImmunoCult T cell expansion media at a concentration of 10⁶ cells/ml. Then 500 ul (˜500,000 cells) of the cell suspension is aliquoted to each well. The cells are then cultured in the presence of CD3/CD28 for 1-2 days prior to addition of virus. Different concentrations of lentiviral particles are added to each well for the desired target MOI. The plates are then sealed with parafilm, and the cells are spun in a table top centrifuge at 300λg for 5 minutes. After spinoculation, the cells are incubated at 37° C. The cells are then assessed for FOXP3 expression and cellular localization and mutant CD25 polypeptide expression.

### Example 2. Mutant CD25 Generation—Binding Affinity to Basiliximab

A set of experiments was performed to generate mutant CD25 polypeptides and measure their binding affinity to basiliximab. In at least some of the cases, the mutant CD25 polypeptide showed increased affinity for basiliximab as compared to a wild type (WT) CD25 polypeptide.

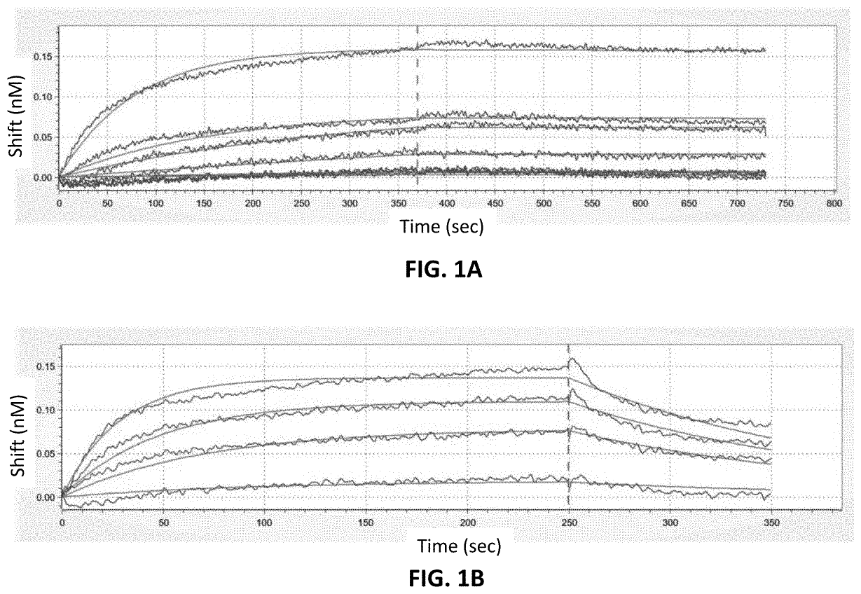

Briefly, binding affinity (K_{D}) of CD25 mutants from Table 1 was measured using biolayer interferometry. Anti-hFc biosensors (GatorBio) were loaded for 5 seconds with 10 μg/mL CD25-Fc mutants followed by acquisition of the baseline signal in phosphate buffer saline supplemented with bovine serum albumin and sodium azide. For the association step loaded probes were incubated for 250 seconds with a titration of basiliximab fab at concentrations generally between 0 nM and 2 μM. This was followed by a dissociation step in buffer lacking basiliximab for 250 seconds. Experimental data was normalized to a reference probe and high-frequency noise was removed by Savitzky-Golay filtering. On and off-rate constants were determined using a globally fitted 1:1 binding model. FIG. 1A and FIG. 1B show exemplary binding affinity data for CD25 L42A (FIG. 1A) and CD25 wild type (WT) (FIG. 1B).

TABLE 1
K_{D} for CD25 mutants binding to Basiliximab fab

| | kD (M) | kon (1/Ms) | koff (1/s) |
|---|---|---|---|
| L42A | 2.42E−10 | 1.03E+05 | 2.49E−05 |
| L42W | 1.04E−09 | 1.94E+05 | 2.01E−04 |
| L2F | 1.07E−09 | 2.36E+06 | 2.52E−03 |
| L42G | 1.14E−09 | 1.33E+05 | 1.52E−04 |
| Y43R | 1.39E−09 | 2.36E+05 | 3.99E−04 |
| L2Y | 1.51E−09 | 1.75E+06 | 2.63E−03 |
| L42Y | 1.75E−09 | 1.86E+05 | 3.25E−04 |
| WT | 3.45E−09 | 2.32E+06 | 8.00E−03 |
| L2I | 3.46E−09 | 2.12E+05 | 7.34E−04 |
| T47D | 3.66E−09 | 2.15E+06 | 7.87E−03 |
| S41N | 3.85E−09 | 1.84E+06 | 7.07E−03 |
| L42V | 3.93E−09 | 1.90E+05 | 7.48E−04 |
| T47S | 4.49E−09 | 1.31E+06 | 5.88E−03 |
| S41Q | 5.25E−09 | 9.75E+05 | 5.12E−03 |
| S41E | 6.08E−09 | 1.18E+06 | 7.19E−03 |
| S41D | 6.49E−09 | 1.09E+06 | 7.08E−03 |
| L42F | 7.65E−09 | 1.01E+05 | 7.72E−04 |
| T47N | 1.28E−08 | 6.08E+05 | 7.76E−03 |
| L42I | 2.33E−08 | 4.99E+05 | 1.16E−02 |

### Example 3. Mutant CD25 Generation—Binding Affinity to hIL2

A set of experiments was performed to generate mutant CD25 polypeptides and measure their binding affinity to hIL2. In at least some of the cases, the mutant CD25 polypeptide showed increased affinity for hIL2 as compared to a wild type (WT) mature CD25 polypeptide.

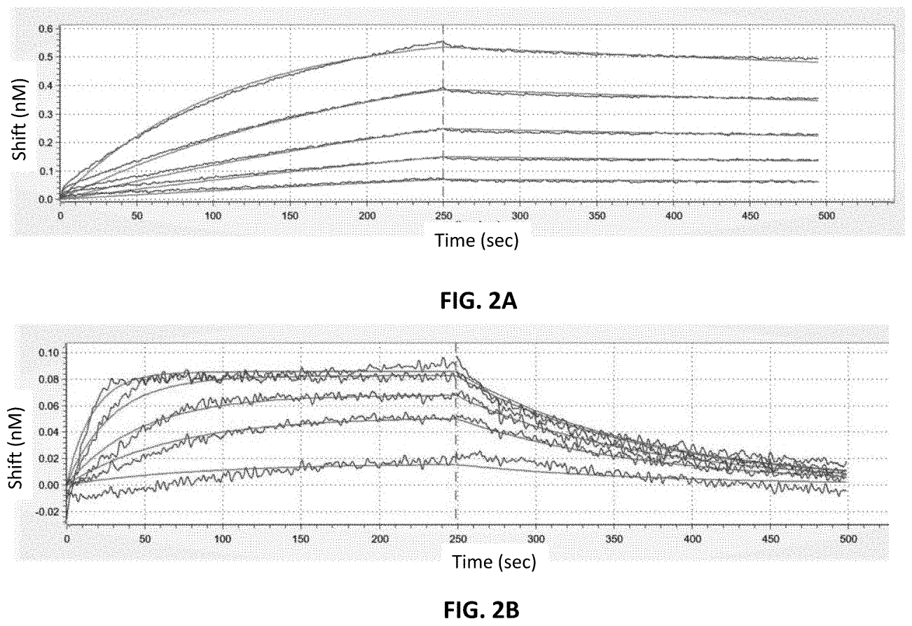

Briefly, binding affinity (K_{D}) of CD25 mutants from Table 2 was measured using biolayer interferometry. Anti-hFc biosensors (GatorBio) were loaded for 5 seconds with 10 μg/mL hIL2-Fc or 10 μg/mL CD25-Fc mutant followed by acquisition of the baseline signal in phosphate buffer saline supplemented with bovine serum albumin and sodium azide. For the association step loaded probes were incubated for 250 seconds with a titration of CD25 mutants (−Fc cleaved) or hIL2 at concentrations generally between 0 nM and 2 μM. This was followed by a dissociation step in buffer lacking CD25 mutants or hIL2 for 250 seconds. Experimental data was normalized to a reference probe and high-frequency noise was removed by Savitzky-Golay filtering. On and off-rate constants were determined using a globally fitted 1:1 binding model. FIG. 2A and FIG. 2B show exemplary binding affinity data using for CD25 WT (FIG. 2A) and CD25 L42A (FIG. 2B).

TABLE 2
K_{D} for CD25 mutants binding to hIL2

| Name | kD (M) | kon (1/Ms) | koff (1/s) |
|---|---|---|---|
| L2I | 1.29E−08 | 7.97E+05 | 1.03E−02 |
| Y43R | 1.38E−08 | 8.93E+03 | 1.23E−04 |
| L42A | 2.30E−08 | 5.27E+05 | 1.21E−02 |
| WT | 2.32E−08 | 2.71E+04 | 6.30E−04 |
| L42V | 2.54E−08 | 6.62E+05 | 1.68E−02 |
| S41Q | 2.54E−08 | 1.14E+04 | 2.88E−04 |
| T47S | 4.07E−08 | 5.82E+05 | 2.37E−02 |
| L42I | 5.86E−08 | 5.77E+05 | 3.38E−02 |
| S41D | 6.03E−08 | 2.03E+04 | 1.22E−03 |
| T47N | 6.88E−08 | 5.20E+05 | 3.58E−02 |
| S41E | 8.22E−08 | 7.67E+03 | 6.31E−04 |
| L2Y | 1.32E−07 | 2.56E+05 | 3.39E−02 |
| L42G | 1.57E−07 | 5.12E+03 | 8.01E−04 |
| L42Y | 3.27E−07 | 2.46E+03 | 8.04E−04 |
| L42W | 1.03E−05 | 1.89E+02 | 1.94E−03 |
| T47D | 1.05E−05 | 1.60E+03 | 1.68E−02 |
| S41N | 5.17E−04 | 1.41E+02 | 7.31E−02 |
| L2F | 5.25E−04 | 5.64E+01 | 2.96E−02 |

### Example 4. Measurement of IL-2 Mediated Survival of Engineered T Cells Compared to Non-Engineered T Cells

A set of experiments is performed to assess IL-2 mediated survival in engineered T cells compared to non-engineered T cells (e.g., natural T regulatory cells and native T cells). Engineered T cells include a first nucleic acid sequence encoding a FOXP3 polypeptide and a second nucleic acid sequence encoding a CD25 polypeptide (e.g., a mutant CD25 polypeptide)). In at least some of the cases, the mutant CD25 polypeptide has increased affinity for basiliximab as compared to a wild type CD25 polypeptide. Basiliximab is an IL-2 agonist which eliminates CD25 expressing cells via blockage of IL2 binding to IL-2Ra subunit, CD25. By preventing IL-2 binding to CD25, basiliximab treatment leads to a decrease in IL-2-mediated cellular responses such as proliferation and cell survival. Therefore, the engineered T cells having a mutant CD25 polypeptide having higher binding affinity to basiliximab than wild type CD25 binds to Basiliximab with a greater affinity as compared to non-engineered T cells, which results in decreased survival of engineered T cells as compared to non-engineered T cells.

Briefly, engineered T cells, natural Tregs cells (CD4⁺CD25⁺CD127^{low} CD45RA⁺) and CD4⁺ naïve T cells (CD4⁺CD25^{low} CD45RA⁺) are purified by magnetic cell sorting system (Miltenyi et al. Cytometry 11: p 231-238, 1990) or a Sony FX Cell Sorter System. Purified cells are cultured in the presence of recombinant human IL-2 (2000 units/mL, Miltenyi, Cat No.: 170-076-148) with or without different concentrations of basiliximab. After 72-hour incubation, proliferation and survival is assessed by flow cytometric analysis of Ki-67 protein, and active caspase 3 plus a fixable viability dye. Cells harvested from the culture are stained first with the fixable viability dye eFluor 780 (eBioscience, Cat No.: 65-085-14), permeabilized using a FOXP3/Transcription factor staining buffer set (eBioscience, Cat No.: 00-5523-00), and then intracellularly stained with nuclear Ki-67 protein and active caspase 3 intracellularly using an PE-conjugated anti-human Ki-67 antibody (eBioscience, Cat No.: 12-5699-41) and Alexa Fluor 647-conjugated anti-caspase 3 antibody (Cell Signaling Technology, Cat. No.: 9602). Comparison of the percentage of Ki-67⁺ cells and cell viability/cell death (Ki-67⁺/caspase 3⁺) between engineered T cells, natural Treg cells, and CD4⁺ naïve T cells shows that basiliximab treatment led to a decrease in cell viability and cell viability/cell death for engineered T cells as compared to non-engineered T cells (e.g., natural T regs and CD3 naïve T cells).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A T cell comprising:
a first nucleic acid sequence encoding a FOXP3 polypeptide; and
a second nucleic acid sequence encoding a cellular suicide agent.

2. The T cell of claim 1, wherein the first nucleic acid sequence encoding a FOXP3 polypeptide comprises a mutation in exon 2, wherein the FOXP3 polypeptide comprising a mutation in exon 2 results in increased nuclear localization of the FOXP3 polypeptide as compared to a FOXP3 polypeptide comprising an exon 2 that does not include a mutation.

3. The T cell of claim 2, wherein the mutation comprises deletion of exon 2.

4. The T cell of claim 1, wherein the cellular suicide agent is a CD25 polypeptide.

5. The T cell of claim 4, wherein the CD25 polypeptide is a mutant CD25 polypeptide, wherein the mutant CD25 polypeptide comprises one or more amino acid substitutions, insertions, or deletions as compared to a wild type CD25 polypeptide.

6. (canceled)

7. The T cell of claim 5, wherein the mutant CD25 polypeptide comprises one or more amino acid substitutions selected from the group consisting of: L42A, L42W, L2F, L42G, Y43R, L2Y, L42Y, L2I, T47D, S41N, L42V, T47S, S41Q, S41E, S41D, L42F, T47N, and L42I.

8. The T cell of claim 7, wherein the mutant CD25 polypeptide comprises a L42A amino acid substitution.

9. The T cell of claim 7, wherein the mutant CD25 polypeptide comprises a L42W amino acid substitution.

10. The T cell of claim 7, wherein the mutant CD25 polypeptide comprises a L2I amino acid substitution.

11. The T cell of claim 7, wherein the mutant CD25 polypeptide comprises a Y43R amino acid substitution.

12. The T cell of claim 7, wherein the mutant CD25 polypeptide comprises a L2F amino acid substitution.

13. The T cell of claim 7, wherein the mutant CD25 polypeptide comprises a L42G amino acid substitution.

14. The T cell of claim 7, wherein the mutant CD25 polypeptide comprises a L2Y amino acid substitution.

15. The T cell of claim 7, wherein the mutant CD25 polypeptide comprises a L42Y amino acid substitution.

16. The T cell of claim 5, wherein the mutant CD25 polypeptide has increased affinity for basiliximab as compared to a wild type CD25 polypeptide.

17. The T cell of claim 1, wherein the first nucleic acid sequence is operably linked to a promoter and the second nucleic acid sequence is operably linked to a promoter.

18. The T cell of claim 1, wherein the T-cell further comprises a third nucleic acid sequence encoding a receptor polypeptide, and wherein the third nucleic acid sequence is operably linked to a promoter.

19. (canceled)

20. The T cell of claim 18, wherein the receptor polypeptide is a chemokine receptor polypeptide comprising one of CCR6, CCR9, or GRP15.

21. (canceled)

22. The T cell of claim 1, wherein the T cell further comprises a nucleic acid sequence encoding a binding agent, wherein the nucleic acid sequence encoding the binding agent is operably linked to a promoter.

23. (canceled)

24. The T cell of claim 22, wherein the binding agent comprises an antigen-binding domain selected from the group consisting of: a Fab, a F(ab′)₂ fragment, a scFV, a scAb, a dAb, a single domain heavy chain antibody, and a single domain light chain antibody.

25. The T cell of claim 22, wherein the binding agent is a chimeric antigen receptor, wherein the chimeric antigen receptor comprises an extracellular domain, a transmembrane domain, and an intracellular domain, wherein the extracellular domain comprises an antigen-binding domain capable of binding to an antigen on an autoimmune cell, and wherein the intracellular domain comprises a cytoplasmic signaling domain and one or more co-stimulatory domains.

26. The T cell of claim 25, wherein the antigen-binding domain is a scFv that is capable of binding to the antigen on the autoimmune cell.

27. The T cell of claim 25, wherein the cytoplasmic signaling domain is a CD3 zeta domain and the co-stimulatory domain comprises at least one of a CD48 domain, a 4-1BB domain, an ICOS domain, an OX40 domain, and a CD27 domain.

28. (canceled)

29. A composition comprising a T cell of claim 1.

30. A method of producing a T-cell population expressing an exogenous FOXP3 polypeptide and a cellular suicide agent, the method comprising culturing a T-cell of claim 1 in growth media under conditions sufficient to expand the population of T-cells.

31.-32. (canceled)

33. A vector comprising:
a first nucleic acid sequence encoding a FOXP3 polypeptide; and
a second nucleic acid sequence encoding a cellular suicide agent.

34.-35. (canceled)

36. The vector of claim 33, wherein the cellular suicide agent is a CD25 polypeptide, wherein the mutant CD25 polypeptide comprises one or more amino acid substitutions selected from the group consisting of: L42A, L42W, L2F, L42G, Y43R, L2Y, L42Y, L2I, T47D, S41N, L42V, T47S, S41Q, S41E, S41D, L42F, T47N, and L42I.

37.-66. (canceled)

67. A method of treating a subject, wherein the method comprising:
administering to the subject a T cell of claim 1; and
after a period of time, administering to the subject an effective amount of a control agent capable of interacting with a cellular suicide agent, wherein the interaction between the control agent and the cellular suicide agent reduces the number of administered T cells in the subject.

68.-76. (canceled)

77. A method of reducing the number of administered T cells in a subject, the method comprising:
administering to the subject an effective amount of a control agent capable of interacting with a cellular suicide agent, wherein the interaction between the control agent and the cellular suicide agent reduces the number of administered T cells in the subject.

78.-81. (canceled)
